SEQUENCE LISTING (41 sequences)

SEQ ID NO: 1 - protein, 371 aa, synthetic construct
  REGION 1..371: note = UPO1 mut
MKYFPLFPTLVYAVGVVAFPDYASLAGLSQQELDAIIPTLEAREPGLPPGPLENSSAKLVNDEAHPWKPLRPGDIRGPCPGLNTLASHGYLPRNGVATPAQIINAVQEGFNFDNQAAIFATYAAHLVDGNLITDLLSIGRKTRLTGPDPPPPASVGGLNEHGTFEGDASMTRGDAFFGNNHDFNETLFEQLVDYSNRFGGGKYNLTVAGELRFKRIQDSIATNPNFSFVDFRFFTAYGETTFPANLFVDGRRDDGQLDMDAARSFFQFSRMPDDFFRAPSPRSGTGVEVVVQAHPMQPGRNVGKINSYTVDPTSSDFSTPCLMYEKFVNITVKSLYPNPTVQLRKALNTNLDFLFQGVAAGCTQVFPYGRD

SEQ ID NO: 2 - protein, 373 aa, synthetic construct
  REGION 1..373: note = UPO 2
MRGAARFAVLIALFTHAAIAFPAYGSLAGLTREQLDEILPTLEIREPGKPPGPLKDTSAKLVNDKAHPWKPVAPADIRGPCPGLNTLASHGWLPRNGIASPSEIITAVQEGFNMDNGLAIFVTYAAHLVDGNILTDKLSIGGKTGLTGPNPPAPAIVGGLNTHAVFEGDTSMTRGDFFFGNNHDFNETLFDEFVDFSNRFGAGKYNLTVAGEFRWQRIQDSIATNPEFSFVSPRFFTAYAESTFPINFFIDGRQTDGQLDLTVARGFFQNSRMPDDFHRANGTRGTEGIDLVAEAHPIEPGSNVGGVNNYVVDPTSADFSTFCLLYENFVNKTVKGLYPNPTGALRKALNTNLGFFFSGISDSGCTQVFPYGK

SEQ ID NO: 3 - protein, 377 aa, synthetic construct
  REGION 1..377: note = UPO 3
MLKLFFVQTALLALSGTTFAYPSHMSLAGLTREQLDQIVPTLTFTPPPPPPAPLNDTSAKLVNDPAHPWQPLRAGDIRGVCPGLNTLASHGYLPRNGIVTPNQIIEAAQDGFNMDNTLARFLAYGTFLVDGNVVTNEMSIGSKSAATGPDPPAPAIVGGLDTHAVFEGDASMTRQDFFFGNNHDFNETLFDQFVEFSNRFGAGKYNLTVAGELRHQRIQQSIATNPNFTFVAPRYFTAFAESAFPVDFFIDGRDSNGQLEMDVARSFFQNSRFPDGFFRPNHSVTGEGSDVVFAAHPIEPGRNVGGVNNYVLDPTSADFTTPCLLYTNFVNETIVGLYPSPTGDLRTALNFYLNLFFEAFDNSEGSGCTQLFPYGQD

SEQ ID NO: 4 - protein, 375 aa, synthetic construct
  REGION 1..375: note = UPO 4
MFSLLNFVTLALACTWSALAFPSSYTSLGGLPREELDRILPSLQYRSPGAPPGPLKFNGTKLVNDDQHPWKPLKHGDMRGPCPGLNTLASHGYLPRNGIATPVQIINAVQEGFNMENSVARLVTYAAHLVDGNLVTDKLSIGGKSPLTGPSPPAPANAAGLNTHALFEGDVSMTRADAFFGDNHSFNETLFDEFTAFSNQFGAGKYNLTVAAEYRFHRIQESIATNPNFSFVSPRFFTAYAESVFPINFFIDGRQGDGQLDLDVARGFFQNMRMPDGFHRASIPTGLEGLAEIASVHPISPGANVNGVNTYTFDPSSADFTTFCLLYVNFVNQTVRSLYPEPTGNLKKALKKNLEFLYGPFSDQCSQVFPYGKDN

SEQ ID NO: 5 - protein, 376 aa, synthetic construct
  REGION 1..376: note = UPO 5
MARLTFLVAVALALSSTTVAFPSYGSLAGLSEAELDRIIPLLEARDACPPPGPLKNTSTKLVNDKDHPWKPLRDGDIRGPCPGLNTLASHGWLPRNGVATPAQIINAVQEGFNMGNDLAVFVTYAAHLVDGNQVTDLLSIGGKTPQTGPDPPQPAIVGGLDTHAVFEGDASMTRGDAFFGDNHSFNETQFDEFSAFSNKFGGGYYNLSVAAEFRWQRIQESIATNPNFSFISPRYFTAYAESVFPLVFFVDGRVSDGRLSLPNARGFFQNSQMPTDFFRPNQSIGLDVIGDGISAIASAHPIAPGKNEGVGNYVLDPTSADFDHFCLLYINFVNQTVKSLYPNPTGVLRDALKRNLDNFYSPLNGSDCVQIFPYGK

SEQ ID NO: 6 - protein, 376 aa, synthetic construct
  REGION 1..376: note = UPO 6
MVQFTVILSLLLATGKALAFPQYGSLAGLSERELEDILPRLHAVKPPPPPGPLNDTSTKLVNNPAHPFLPQRNGDMRGPCPGLNTLASHGYLPRNGIATPAQIINAVQEGFNMGNDLAVFVTYAAFLVDGNQVTNLLSIGGKSSLTGPDPPKPAIVGGLDTHAVFEGDASMTRGDAFFGDNHSFKENQFDEFIAFSNKFGGGKYNLTVASEFRWQRIQESTATNPNFSFISPRYFTAYAESTFPITFFVDGRNEDGALSLDVARGFFQDSRMPNGFFRANQSIGLDIIGSLIDFIFEPHPIQPGGNQGRVNSYTVDPNSANFSQFCQLYQDFVNNTVKGLYPNPQGVLRDNLNTNLGFFFSPLQGSGCPQVFPFGQ

SEQ ID NO: 7 - protein, 376 aa, synthetic construct
  REGION 1..376: note = UPO 7
MRFFSHLSIIPLLSLHGVLAFPSYGTLAGLSRSELEAILPTLKPGVPESPPGPLNDTSAKLVNDKKHPWKPAGKKDIRGPCPGLNTLASHGWLPRDGVATPAQIVNAVQEGFNMGNDLAVFVTYAAHLVDGNLITNLLSIGGKTDRTGPNPPPPAIVGGLNTHAVFEGDASTTRADFFFGDNHSFNETLFDELTAFSNKFGGGFYNLSVAAEFRFQRIQDSIATNPQFDFISPRYFTAYAESIFPLTFFIDGRDKSLHLDMNVARGFFQNSRFPDGFFRSNTSITLDVIGGGIDYIFSKHPVPPGSNNGTVNSYTPNPNSADFTQFCKLYTDFVNITIRGLYPNAKGALLTALNKNLEYFYSPLVGSGCPQVPPFV

SEQ ID NO: 8 - protein, 376 aa, synthetic construct
  REGION 1..376: note = UPO 8
MARVFFAIAALLLAAKDVVSFPNYASLAGLSERELDEIIPQLTVRTLEKPPGQMKNTLTKLVNDPAHPWIAPAPDDQRDPCPGLNTLANHGYLPRDGIATPAQIVNAVQEGFNMANDIAVFVTYAAHLVDGNLLTDLLSIGGKSAKTGPNPPSPAIVGGLDTHAVFEGDASTTRGDAFFGDNHSFNESLFDELTAFSNKFGAGFYNLSVATEFRFQRIQDSIATNPQFSLISPRYYTAYAESVFPVAFFVDGRETNGSLNMTVARGFFQDGRMPNDFFRSNISWGLDLIGEGIGFIFTPHPIEPGTNNGTLNSYTLDPNSADFSDFCKLYTDFVNVTVRGLYPNATGPLLNALNQNLDFFFGPLGDQGCTQVPAFV

SEQ ID NO: 9 - protein, 275 aa, synthetic construct
  REGION 1..275: note = UPO 9
MKLNIFSTTLAIGLVSAGAHYHQQDVVANGTEGVWIAPTDTDYRGPCPMMNTLANHGFLPRDGKNLTEYNVVKGLNDGLNFNKSLATIMFQQAIPASPAYPNATFFTLNDLNRHNVLEHDGSISRSDAYYGNNYIFNQTIFDTTKAYWPSETLTAQHLIDGKMFRQIVSRSTNPNYTFSATTQQFSLGEMAAPIVAFGDKYVVTANRTLVESWIENERLPTELGWRKPVEEILLSDITYVTEVLGNLTSLYSTVIITPNPDSLAKRQMGHWGQSI

SEQ ID NO: 10 - protein, 265 aa, synthetic construct
  REGION 1..265: note = UPO 10
MKTTTLLCLAAALTQTYAFPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGEMDFRATIALPRLVYIYYEEA

SEQ ID NO: 11 - protein, 263 aa, synthetic construct
  REGION 1..263: note = UPO 11
MKTTTLLFLVGALTQTHAFPQQGVPHPLPWSPPGPNDVRAPCPMLNTLANHGFLPHNGKNITQQHTINALYNALGIDAELATYLHQEAVTTNPVPNATTFSLNDLSRHDILEHDASLSRQDAFFGDNHDFNQTIFNQTRSYWTSPIIDVKQAALARQARVNTSMATNPNYTMSELGDAFSYGETAAYIIVLGDKEAGLVNRSRVEYLFENERLPVELGWSRARENITFDDLSTMLNKIINATGGESEFERELAKRGGVHVGWR

SEQ ID NO: 12 - protein, 261 aa, synthetic construct
  REGION 1..261: note = UPO 12
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGDTRCPYGYH

SEQ ID NO: 13 - protein, 283 aa, synthetic construct
  REGION 1..283: note = UPO 13
MKTLFLLTLAAFTPVFAGFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEIALKTQPSTPSIKPTQTPSSPTRLLLKRLGRQLMLIVPRPIRLRVLRNTPPLRLITKNKPREMAPNLLILAVHKRATSMQKR

SEQ ID NO: 14 - protein, 225 aa, synthetic construct
  REGION 1..225: note = UPO 14
MRTSLLPALAAVSPVLAGFDTWAPPGPYDVRGPCPMLNTLTNHGFFPHDGQDIDRETTENALFDALHVNKTLASFLRADAYHGSVLAFNHTIFEETKSYWTDETVTLKMAADARYYRIKSSQATNPTYQMSELGDAFTYGESAAYVVLFGDKESQTVPRSWVEWLFEKEQLPQHLGWKRPATSFELNDLDKFMALIQNYTQEIEEPSCESRKQRRKPRGPSHFGF

SEQ ID NO: 15 - protein, 375 aa, synthetic construct
  REGION 1..375: note = UPO 15
MAKFSTLFAFSALAIQAIALPQYRSLAGLSERELEGILPRLNVVTPPPSPPGPPNDTSVKLVNDAAHPFMPLQDGDIRGPCPGLNTLASHGYLPRNGIATPAQIINAVQDGFSMDNGLATLLAYATMLVDGNPLTNLMSIGGKSPLTGMDPPQPAIVGGLDTHAVFEGDASMTRADFFFGDNHSFNQTLFNQFANFSNQFGDGNYNLTTAEEYRFFRIQQSIAENPQFSFISPRFFTAYFESAFPLVFFVDGRQADGQLSVENATSFFRDMQFPDDFHRADGSQTADLVNNAATAIFSAHPMQPGGNNGTVNSYTFDPNSANFTEGCKLYTDFVNNVVVPLYPTPQGALKVNLNANLGFLFSTFSNCTQVFPYGQ

SEQ ID NO: 16 - protein, 371 aa, synthetic construct
  REGION 1..371: note = UPO 16
MAKFSTLLALSVLAIQAVAFPQHQPLAGLTERELEDLLPRFKPVVPPPPPGPPKDTSVKLVNDKDHPYEPLRKGDIRGPCPGLNTLASHGYLPRNGVVTPAQIINAVQDGFGMDNELAILLAYSTMLTDGNVVTNLMSIGQKTPLTGPDPPAPAIVGGLNTHGTFEGDAGLTRADFFFGDNHSFNQTLFNEFVEFSNKFGGGVYNQTVAAEYRFFRIQQSTAENPTFTFVTPRFVTAYRESVFPFIFFVDGRKADGQLSMKDAFGFFNESRMPDGFHRADGSKTADLVGNASDAIFAAHPVQPGANAGKVNTYTPDPNSPTDDCGLYETFVNLMVKQYPNPQGVLRTNLNLNLGFFFQGFPGCTQLFPFGQ

SEQ ID NO: 17 - protein, 268 aa, synthetic construct
  REGION 1..268: note = UPO 17
MLGIRLVSLLAFTGSALAELDFSKWKTRQPGELRAPCPAMNSLANHGFIQRDGKNITVEGLTPVLKEVFHLSHELAFTVSQLGLFTALDPSKGVFTLQDLTDRHNVFEHDASLSREDAKFGGDQSVLHKGQFQKFMDHFKGEKYISFEAAAKARYAMVQDSRKRNPDFTYDVTHRITSYGETIKYLRTIVEPSTGKCPVDWIKILFEQERLPYNEGWRPPTNELSGFSLASEVLELALITPEKLPVDECLGKGKGKGNCKRRRSYLGI

SEQ ID NO: 18 - protein, 371 aa, synthetic construct
  REGION 1..371: note = UPO 18
MNPFLKLAVLALVTAPLAGAFPSHRSLGGLSSEQLDRIFPTLKVAPPEGPPPPQDDTSTRLVDDADHPFMPAGPNDMRGPCPGLNTLASHGYLPRNGIATPAQVINATMQGFNMEFSLAKFVTYAAFLVDGNPITNLMSIGGKSDLTGEDPPDPATVGGLNTHAVFEGDASMTRADAFFGDNHSFNQTLWDGFVDFSNRFGAGKYNLTVATELRIQRIQDSIATNPQFSFVSPRFITAYAESTFPINFFIDGRQQDGQLDLDAAISFFRDMRYPSGFFRAPKPMGVEGIETIIAAHPIPAGANNGAVNTYTPDPHSGDFNSFCTVYTNFVNETIRGLYPSPTGILKDSLNRNLDFLHDFVSGCPQIFPWGR

SEQ ID NO: 19 - protein, 321 aa, synthetic construct
  REGION 1..321: note = CPO 19
MFSKVLPFVGAVAALPHSVRQEPGSGIGYPYDNNTLPYVAPGPTDSRAPCPALNALANHGYIPHDGRAISRETLQNAFLNHMGIANSVIELALTNAFVVCEYVTGSDCGDSLVNLTLLAEPHAFEHDHSFSRKDYKQGVANSNDFIDNRNFDAETFQTSLDVVAGKTHFDYADMNEIRLQRESLSNELDFPGWFTESKPIQNVESGFIFALVSDFNLPDNDENPLVRIDWWKYWFTNESFPYHLGWHPPSPAREIEFVTSASSAVLAASVTSTPSSLPSGAIGPGAEAVPLSFASTMTPFLLATNAPYYAQDPTLRPQRQA

SEQ ID NO: 20 - protein, 376 aa, synthetic construct
  REGION 1..376: note = CPO 20
MFSKILPLVGVAAALPHWLQLRQEPNSGIGYPYDNHTKPYVHPGPHDSRAPCPALNALANHGYIPHNGRAITKENLQNAFLEHMGIGNSVIALALTNAFVVCEYVTGQDCGDTLVNLTLLSEPHAFEHDHSFSRKDYKQGVSNFNEIVDNRNFDLSTFETSLDVVAGQTHFGYAEMNQIRLQRESLSNEADFPGWFTESKPIQEVEAGFIFALVSDFNLPDNDENPLVRVDWWKYWFINESFPYHLGWHPPTPAREIEFVTSASSAILAAAVTSTPSSLPSGAIGPGAEAVPLSFASTMTPFLLATDIPYFAHPTLGPNDKREAAPAPAATTSTATFKNPYLEPIGTQDIKNQQAYVSSKAAAMSSAMAVNKARSL

SEQ ID NO: 21 - protein, 371 aa, synthetic construct
  REGION 1..371: note = UPO 21
MKYFPLFPTLVFAARVVAFPAYASLAGLSQQELDAIIPTLEAREPGLPPGPLENSSAKLVNDEAHPWKPLRPGDIRGPCPGLNTLASHGYLPRNGVATPVQIINAVQEGLNFDNQAAVFATYAAHLVDGNLITDLLSIGRKTRLTGPDPPPPASVGGLNEHGTFEGDASMTRGDAFFGNNHDFNETLFEQLVDYSNRFGGGKYNLTVAGELRFKRIQDSIATNPNFSFVDFRFFTAYGETTFPANLFVDGRRDDGQLDMDAARSFFQFSRMPDDFFRAPSPRSGTGVEVVIQAHPMQPGRNVGKINSYTVDPTSSDFSTPCLMYEKFVNITVKSLYPNPTVHVRKALNTNLDFFFQGVAAGCTQVFPYGRD

SEQ ID NO: 22 - protein, 262 aa, synthetic construct
  REGION 1..262: note = UPO22
MKLVYLSSAVAFGSAIADTAPWEGPGPNDVRGPCPMLNTLANHGFLPHDGKNIHVNKTVDALSSALNIDPELGSFLHSFAVTANPQPNATWWNLDHLSRHNILEHDASLSRQDAYFGAPDVFNEAVFNQTKSYWTGDVITLQMAANARLARLMTSNLTNPEYSMSDLGSSFSIGESVAYVAILGSKETRTVPKAYVEYLFEKERLPYELGFKKAETPMTETDLGNLMDELISLQHFPQSPGKIAKRSERPSEKRAEKRCPFH

SEQ ID NO: 23 - protein, 267 aa, synthetic construct
  REGION 1..267: note = UPO23
MKTATLLFLAAGLTQTQAFPSQGAAPHPLPWSPPGPNDVRAPCPMLNTLANHGYLPHNGKNITEQHTINALYNALGIDAELSAFLHQEAVTTNPTPNATTFSLNDLSRHDILEHDASLSRQDAYFGDNHDFNQTIFDETRSYWTSPIIDVKQAALSRQARVNTSMATNPNYTMSELGASFSYGETAAYIIVLGDKENGLVNRSRVEYLFENERLPLDLGWTRAKENITFDDLRTMLNRIVNATGGESEFDRELAKRGGVHVGRWRGY

SEQ ID NO: 24 - protein, 265 aa, synthetic construct
  REGION 1..265: note = UPO24
MKTTTLLCLAAALTQTYAFPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGESEFDRELAKRGGVHVGSWRG

SEQ ID NO: 25 - protein, 267 aa, synthetic construct
  REGION 1..267: note = UPO25
MKTTPLLFFAAGLAQTHAFPSQGGAPHPLPWSPPGPNDVRAPCPMLNTLANHGYLPHNGKDITEQHTINALYNALGIDAELATYLHQEAVTTNPAPNATTFSLNDLSRHDILEHDASLSRQDAFFGDNHDFNQTIFDETRSYWTSPIIDVMQAALSRQARVDTSMATNPNYTMSELGASFSYGETAAYIIVLGDKENGLVNRSRVEYLFENERLPLDLGWTRAKENITFDDLSTMLNRIVNATGGESEFDRELAKRGGVHVGKWRGY

SEQ ID NO: 26 - protein, 60 aa, synthetic construct
  REGION 1..60: note = Conserved Motif
YDNNTLPYVAPGPTDSRAPCPALNALANHGYIPHDGRAISRETLQNAFLNHMGIANSVIE

SEQ ID NO: 27 - protein, 59 aa, synthetic construct
  REGION 1..59: note = Conserved Motif
VNDKDHPWKPLRPGDIRGPCPGLNTLASHGYLPRNGVATPAQIINAVQEGFNMDNSVAL

SEQ ID NO: 28 - protein, 48 aa, synthetic construct
  REGION 1..48: note = Conserved Motif
LALTNAFVVCEYVTGSDCGDSLVNLTLLAEPHAFEHDHSFSRKDYKQG

SEQ ID NO: 29 - protein, 60 aa, synthetic construct
  REGION 1..60: note = Conserved Motif
FATYEAHLMVGNLLTDLLSIGRKTPLTGPDLPPPANIGGLSEHGLFEGDASMTRGDAFFG

SEQ ID NO: 30 - protein, 261 aa, synthetic construct
  REGION 1..261: note = UPO 12 variant
MKSLSFSLALGFGSTLVYSAPSPFSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGDTRCPYGYH

SEQ ID NO: 31 - protein, 261 aa, synthetic construct
  REGION 1..261: note = UPO 12 variant
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIYVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGDTRCPYGYH

SEQ ID NO: 32 - protein, 261 aa, synthetic construct
  REGION 1..261: note = UPO 12 variant
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGDTRSPYGYH

SEQ ID NO: 33 - protein, 255 aa, synthetic construct
  REGION 1..255: note = UPO 12 variant
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGDTR

SEQ ID NO: 34 - protein, 261 aa, synthetic construct
  REGION 1..261: note = UPO 12 variant
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGNTRCPYGYH

SEQ ID NO: 35 - protein, 261 aa, synthetic construct
  REGION 1..261: note = UPO 12 variant
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKVEKRGITRCPYGYH

SEQ ID NO: 36 - protein, 248 aa, synthetic construct
  REGION 1..248: note = UPO 12 variant
MKSLSFSLALGFGSTLVYSAPSPSSGWQAPGPNDVRAPCPMLNTLANHGFLPHDGKGITVNKTIDALGSALNIDANLSTLLFGFAATTNPQPNATFFDLDHLSRHNILEHDASLSRQDSYFGPADVFNEAVFNQTKSFWTGDIIDVQMAANARIVRLLTSNLTNPEYSLSDLGSAFSIGESAAYIGILGDKKSATVPKSWVEYLFENERLPYELGFKRPNDPFTTDDLGDLSTQIINAQHFPQSPGKV

SEQ ID NO: 37 - protein, 266 aa, Hypoxylon sp.
MKSVQLSALIAFGAKAVYSFPSANAPWSGPGTDDVRGPCPMLNTLANHGFLPHSGKGITVNKTIDALNAGLNMEADLAALLFDFAVTTNPTPNASYFDLDHLTRHNILEHDASISRQDSYFGRADILNEAVFNQTKSYWTGELVDIQMAANARVARLMTSNLTNPEYSLSDTGSVFSIGESAAYVGILGDKVSGTVPKTWLIYLFEQERLPYELGFKRPVDPFTEDDLFNMSEAIRDAQHFPQDIGKVTKRGNKARCPHGYCIEVL

SEQ ID NO: 38 - protein, 250 aa, Rosellinia necatrix
MKLTTLLFPAVVLGAACPYGTFKPEEPTDTRGVCPMLNALANHGFLPRDGRNINENQTVTALNNALNLTPDFGRFLFTAGRLSNPKPNSTTFDLNHLDRHNLFEHDGSLSRQDAHFGQWSRFNQTVWNWTMQYWTGDILDVQMVANGRAQRHTRSNLTNPDYALSVVGYDFSVAENAALLSIIGDKVTQTCPKKFVDYLFVNEELPYSVGWKKSELPIALEDLIRTFRDIELATAFPAPPPPDNSGEIFA

SEQ ID NO: 39 - protein, 263 aa, Daldinia sp. EC12
MKLTFMSSVVTLGSAVAAYPTSWEAPGPNDVRGPCPMLNTLANHGFLPHDGKNINVNNTAEALSKGLNLAWELGVDLHDFAVMANPQPNATTFDLDHLSRHNVLEHDGSLSRQDAHFGPPDVFNEAVFNQTVSYWTGDVVTMQMAANARLARLMTSNLTNPEYSLSDLGSGFSIGESVVYLLVLGNKDTAEAPKNYLEYWFRNERLPYELGWERPNVIMTGDDLGNAMDKLVTLQHFPQSPGKITSDPEKASAKLAGKRHLFH

SEQ ID NO: 40 - protein, 265 aa, Monosporascus sp.
MKFELAATILAAGTASAFRLKARDTYDWHPPAYGDVRGPCPMLNTLANHGYLPRNGKDITENRTIEALGTALSIDSELAQLLFEQAITTNPAPNATTFSLNDLVRHNILEHDASLSRVDFYFGNPQPFNQTVFAQTRSYWTTPIIDVQQAANARWARVETSNATNPNFTLSTLGERFSYGESAAYIVILGNKITGTVPRDWVEYLFENERLPLEIGWTRRTGSITRNDLEDVMQQIYAATPNNNATTNSWRGNPRALHMAVRASA

SEQ ID NO: 41 - protein, 273 aa, Aspergillus aculeatus
MRYFVLACAPLLYAVTLAFPRADYVSEGKLPAGHPPLDWKPAGLGDARAPCPMLNTLANHGYLPHDGKDITKAHTIAALHSALNIDRELAQYLFQEALTTNPAANATTFSLNDLSRHNILEHDASLSRLDYYFGDNHDFNQAIFDQTRQHWPDPIITVQAAANAREARVRTSNATNPTFTLSELGTAFGYGETAAYIIILGNKTSGLVDRSWVEYLFENERLPVELGWTRHEEAVSMDDLEGMMQEVINATGHAEEVKRELVRRGDLHVGRRA

(19) United States
(12) Patent Application Publication
(10) Pub. No.: US 2026/0250645 A1
(43) Pub. Date: Aug. 27, 2026

(54) RECOMBINANT HEME THIOLATE OXYGENASES

(71) Applicant: bisy GmbH, Hofstätten an der Raab (AT)

(72) Inventors: Kay Domenico NOVAK, Graz (AT); Anton GLIEDER, Graz (AT); Astrid WENINGER, Graz (AT); Christoph REISINGER, Graz (AT); Claudia RINNOFNER, Graz (AT); Carsten PICHLER, Graz (AT)

(21) Appl. No.: 19/312,071

(22) Filed: Aug. 27, 2025

Related U.S. Application Data
(63) Continuation of application No. 17/625,081, filed on Jul. 6, 2020, now Pat. No. 12,415,991.

(30) Foreign Application Priority Data
Jul. 5, 2019 (EP) 19184720.1

(51) Int. Cl. C12N 9/08; C12N 15/81

(52) U.S. Cl. CPC C12N 9/0065; C12N 15/815; C12N 2830/00; C12Y 111/02001

(57) ABSTRACT

The invention relates to polypeptides having peroxygenase activity and compositions comprising such polypeptides. The invention also relates to improved methods of producing such polypeptides in yeasts.

### CROSS-REFERENCE TO RELATED APPLICATIONS

This application is a continuation of U.S. patent application Ser. No. 17/625,081, filed on Jan. 5, 2022, which is the U.S. national stage of International Patent Application No. PCT/EP2020/069020, filed on Jul. 6, 2020, which claims the benefit of priority under 35 U.S.C. § 119 from European Patent Application No. 19184720.1, filed on Jul. 5, 2019. The disclosures of the foregoing applications are incorporated herein by reference in their entirety.

### SEQUENCE LISTING

The entire content of a Sequence Listing titled “Sequence_Listing.xml,” created on Aug. 27, 2025 and having a size of 60,844 bytes, which has been submitted in electronic form in connection with the present application, is hereby incorporated by reference herein in its entirety.

### DESCRIPTION

### Field of the Invention

The present invention relates to recombinant polypeptides having peroxygenase activity, their encoding polynucleotides, expression vectors and recombinant host cells comprising such polynucleotides or vectors. The present invention also relates to the use of the recombinant polypeptides as catalysts.

### Background Art

In the field of synthetic chemistry is oxygenation of organic molecules one of the major tasks. Oxygen-transferring enzymes can be used to solve this task through biocatalysis. In addition to cytochrome P450 enzymes, flavin dependent monooxygenases or di-iron dioxygenases, unspecific peroxygenases, called UPOs, or also POX due to their peroxidase activity, have the ability to transfer oxygen selectively to a wide range of substrates, such as polycyclic aromatic hydrocarbons, heterocycles, benzene derivatives, alkenes as well as linear and cyclic alkanes. Other reactions catalyzed by UPOs include double bond epoxidations, dealkylations, oxidation of inorganic halides as well as organic hetero atoms and also typical peroxidase reactions including radical based polymerization. Further they can even use pesticides or complex drug molecules as substrate. As such, applications of UPOs can be numerous; they reach from pharmaceutical production to environmental applications, including environmental problems caused by industry. For example, transformation of pollutants through peroxidases can result in reduction of toxicity or bioavailability. Also, removal of pollutants from water can be achieved.

UPOs belong to the peroxidase-peroxygenase group with haloperoxidases such as the Caldariomyces fumago chloroperoxidase (CfuCPO) as first and long known representative. Recombinant production of CfuCPO is also possible in suitable hosts such as Aspergillus spp., but no successful recombinant CfuCPO expression in yeast was published so far. CfuCPO has been the only heme-thiolate peroxidase characterized on the protein level for almost 50 years, before more versatile heme thiolate peroxidases were discovered. In the past decade a new subgroup of enzymes accepting especially aromatic substrates was described. One typical example for aromatic substrate conversion is the formation of 1- and 2-naphthol with naphthalene as substrate. 1-naphthol plays an important role in the production of pharmaceuticals, herbicides, and others. The first aromatic peroxygenase (AaeUPO) described was derived from the mushroom Agrocybe aegerita oxidizing similar substrates as CfuCPO, typical peroxidase substrates and aromatic alcohols and aldehydes. AaeUPO has the unique ability to epoxidize and hydroxylate aromatic rings efficiently by using hydrogen peroxide as oxygen donor.

In spite of the high technological potential and interest in these new secreted heme thiolate enzymes, their recombinant expression showed to be challenging and largely unsolved. Bormann et al. (2015) reported that attempts to express CfuCPO in Escherichia coli, S. cerevisiae, or Pichia pastoris (Komagataella phaffii) did not yield active enzyme. Although recombinant expression was successful in Aspergillus niger, the enzyme levels of few mg/L were significantly lower than with the native host. Molina-Espeja and M. Alcalde (2014) for the first time reported recombinant expression and engineering of AaeUPO in S. cerevisiae, still with very low enzyme yields of less than 0.01 mg/L but the first successful overexpression of a fungal heme thiolate peroxygenase by a yeast species. Employing the native signal sequence of AaeUPO1 resulted in 2-times higher secretion efficiency than the signal sequence of the S. cerevisiae mating factor alpha. No other UPOs could be functionally expressed as secreted enzymes by any yeast species and in general very few recombinant UPOs (expressed by filamentous fungi) were known so far. Directed evolution of AaeUPO1 (also named UPO1) in S. cerevisiae resulted in a mutant enzyme with increased activity and higher recombinant production yields (up to 217 mg/L in P. pastoris). This mutated sequence was also the first UPO which was successfully expressed by P. pastoris (Molina et al (2015)) using a methanol inducible AOX1 promoter and secreted to the culture supernatant. The AOX1 promoter is not a derepressed promoter and relies on methanol to obtain significant expression levels. Without the addition of methanol, the AOX1 promoter shows far less than 1% activity. No other UPO had been overexpressed by P. pastoris before and no natural heme thiolate peroxygenase with UPO or CPO activity had been successfully expressed by P. pastoris.

WO2008/119780 discloses polypeptides having peroxygenase activity. The polypeptides may be produced recombinantly in Aspergillus oryzae.

Thus, there is still the need for an effective expression system for producing novel unspecific peroxygenase enzymes (UPOs) in high yields and high enzyme activity.

### SUMMARY OF INVENTION

It is the objective of the present invention, to provide novel recombinant polypeptides with peroxygenase activity, showing at least complementary activities and properties to known native UPOs and the UPO1 variants developed by Molina et al (2015).

It is further a specific objective of the present invention to provide polypeptides and polypeptide preparations having increased peroxygenase activity compared to the respective native UPOs, and to provide means and methods of their production in yeast cells.

The problem is solved by the present invention.

According to the invention, there is provided a method for producing a polypeptide having peroxygenase activity, comprising:
a. cultivating a yeast cell in a medium conducive for the production of said polypeptide, wherein the yeast cell comprises a polynucleotide comprising a nucleic acid sequence encoding said polypeptide operably linked to a derepressed promoter sequence which is functional in methylotrophic yeasts, and
b. isolating said polypeptide from the cultivation medium.

Specifically, the derepressed promoter sequence is a methanol-independent promoter.

A further embodiment relates to the method as described herein, wherein said promoter is an engineered or synthetic promoter variant.

A further embodiment relates to the method as described herein, wherein the promoter is a CTA1 (PDC) or FMD promoter.

A further embodiment relates to the method as described herein, wherein the expression and/or secretion is increased by co-expression of helper proteins.

A further embodiment relates to the method as described herein, wherein the helper protein is protein disulfide isomerase (PDI).

A further embodiment relates to the method as described herein, wherein said yeast cell is a Pichia pastoris (Komagataella phaffii) cell.

A further embodiment relates to the method as described herein, wherein said polypeptide is obtained in a yield of about 1 mg/L, 10 mg/L, 50 mg/L, or of about 100 mg/L, or of about 250 mg/L.

Specifically, employing the method described herein, said polypeptide having peroxygenase activity, specifically a heme thiolate peroxygenase such as any of the unspecific peroxygenases (UPOs) described herein, is expressed at a yield of at least 250 mg/L.

A further embodiment relates to the method as described herein, wherein said polypeptide is obtained in the culture supernatant in a titer of about 300 mg/L, or of about 0.5 g/L, or of about 1 g/L.

A further embodiment relates to the method as described herein, wherein the polypeptide having peroxygenase activity comprises an MF-alpha signal sequence (“mating factor alpha” signal sequence).

Further provided herein is a method of producing a polypeptide having peroxygenase activity, comprising:
a. cultivating a methylotrophic yeast cell, preferably Pichia pastoris, in a medium conducive for the production of said polypeptide, wherein the yeast cell comprises a polynucleotide comprising a nucleic acid sequence encoding said polypeptide operably linked to a promoter sequence which is functional in methylotrophic yeasts, and
b. isolating said polypeptide from the cultivation medium.

Specifically, said promoter is an engineered or synthetic promoter variant. Specifically, said promoter is a CTA1 (PDC), FMD or AOX1 promoter.

Specifically, expression and/or secretion of the polypeptide having peroxygenase activity is increased by co-expression of helper proteins, preferably PDI.

Specifically, the polypeptide having peroxygenase activity comprises a MF-alpha signal sequence (“mating factor alpha” signal sequence).

Specifically, the yeast cell is a Pichia pastoris (Komagataella phaffii) cell.

One embodiment of the invention relates to a polypeptide which has a peroxygenase activity which is obtained by a method as described herein.

One embodiment of the invention relates to a polypeptide having peroxygenase activity selected from the group consisting of a polypeptide comprising an amino acid sequence having at least 70% sequence identity to the polypeptide of SEQ ID NO:1 (UPO1 mut), SEQ ID NO:2 (UPO2), SEQ ID NO:4 (UPO4), SEQ ID NO: 5 (UPO5), SEQ ID NO:7 (UPO7), SEQ ID NO:11 (UPO11), SEQ ID NO: 12 (UPO12), SEQ ID NO: 17 (UPO17), SEQ ID NO: 18 (UPO18), SEQ ID NO:19 (UPO19), SEQ ID NO:22 (UPO22), SEQ ID NO:23 (UPO23), SEQ ID NO:24 (UPO24), or SEQ ID NO: 25 (UPO25).

One embodiment of the invention relates to a polypeptide having peroxygenase activity comprising an amino acid sequence having at least 75%, 80%, 85%, 90%, 95%, 97%, 98%, or 99% identity to the polypeptide of SEQ ID NO:1 (UPO1 mut), SEQ ID NO:2 (UPO2), SEQ ID NO:4 (UPO4), SEQ ID NO:5 (UPO5), SEQ ID NO: 7 (UPO7), SEQ ID NO:11 (UPO11), SEQ ID NO: 12 (UPO12), SEQ ID NO: 17 (UPO17), SEQ ID NO: 18 (UPO18), SEQ ID NO: 19 (UPO19), SEQ ID NO:22 (UPO22), SEQ ID NO:23 (UPO23), SEQ ID NO:24 (UPO24), or SEQ ID NO:25 (UPO25).

One embodiment of the invention relates to a polypeptide having peroxygenase activity comprising or consisting of the amino acid sequence of SEQ ID NO: 1 (UPO1 mut), SEQ ID NO:2 (UPO2), SEQ ID NO:4 (UPO4), SEQ ID NO:5 (UPO5), SEQ ID NO:7 (UPO7), SEQ ID NO:11 (UPO11), SEQ ID NO:12 (UPO12), SEQ ID NO: 17 (UPO17), SEQ ID NO: 18 (UPO18), SEQ ID NO: 19 (UPO19), SEQ ID NO: 22 (UPO22), SEQ ID NO:23 (UPO23), SEQ ID NO:24 (UPO24), or SEQ ID NO:25 (UPO25).

One embodiment of the invention relates to a polypeptide comprising an amino acid sequence having at least 70% sequence identity to the polypeptide of SEQ ID NO: 12 (UPO12).

One embodiment of the invention relates to a polypeptide as described herein having increased peroxygenase activity when compared to a control peroxygenase (SEQ ID NO:1 (UPO1)), wherein the activity is about 10-fold, 20-fold, or 50-fold when measured in an ABTS assay.

One embodiment of the invention relates to the use of a polypeptide having peroxygenase activity as defined herein as peroxygenase, specifically as a catalyst in organic synthesis processes, polymerization processes, drug metabolite production, environmental application, application in consumer products,

One embodiment of the invention relates to a recombinant polypeptide heaving peroxygenase activity and peroxidase activity, wherein the ratio between peroxidase activity and peroxygenase activity is about 1:1, 1:2, 1:3, 1:4, or 1:5 when peroxidase activity is expressed as ABTS units and peroxygenase activity is expressed as naphthalene units.

One embodiment of the invention relates to a recombinant polypeptide heaving peroxidase activity, with said peroxidase is active in a broad range of pH activity as determined by an ABTS assay.

One embodiment of the invention relates to a recombinant polypeptide heaving peroxygenase activity and peroxidase activity, wherein the KM value for hydrogen peroxide is about 1 mM or lower.

One embodiment of the invention relates to a polypeptide having peroxygenase activity and comprising an amino acid sequence having at least 70%, 75%, 80%, 85%, 90%, 95%, 97%, 98%, or 99% sequence identity to the polypeptide of SEQ ID NO:12 and comprising at least one amino acid modification, and wherein the polypeptide has increased peroxygenase activity when compared to UPO12 (SEQ ID NO: 12). Preferably, said modification is at least one amino acid substitution in the sequence of SEQ ID NO:12. Specifically, the peroxygenase activity is about 1.0-fold, 1.1-fold, 1.2-fold, 1.3-fold, 1.4-fold, 1.5-fold, 1.6-fold, 1.7-fold, 1.8-fold, 1.9-fold, or 2.0-fold or more increased when measured in an ABTS assay and/or 2,6-DMP assay.

A specific embodiment of the invention relates to a polypeptide having peroxygenase activity and comprising an amino acid sequence having at least 70% sequence identity to the polypeptide of SEQ ID NO:12 and comprising one or more amino acid substitutions in the C-terminal region of SEQ ID NO:12 ranging from positions 130 to 261, preferably positions 145 to 261, of SEQ ID NO: 12, wherein the polypeptide has increased peroxygenase activity when compared to UPO12 (SEQ ID NO: 12).

Specifically, provided herein is a modified unspecific peroxygenase (UPO) comprising an amino acid sequence having at least 70% sequence identity to the polypeptide of SEQ ID NO: 12 and having increased peroxygenase activity as compared to the unmodified wild-type UPO12, wherein the modification is a modification of at least one amino acid corresponding to any one of amino acids 145-261 of the unspecific peroxygenase of SEQ ID NO:12.

Specifically, the modified unspecific peroxygenase comprises a sequence having at least about 70%, 75%, 80%, 85%, 90%, 95%, 97%, 98%, or 99% identity to SEQ ID NO:12.

According to a specific embodiment, the modification is a modification of at least one amino acid corresponding to any one or more of amino acids C256, D253, E249, and/or D145 of the unspecific peroxygenase of SEQ ID NO:12. Specifically, the modified UPO comprises at least a mutation corresponding to C256S, D253N, D2531, and/or D145Y.

According to a further specific embodiment, the modification comprises introduction of a stop codon, preferably by an amino acid substitution, and/or comprises deletion of one or more amino acids, preferably at the C-terminus. Specifically, introduction of a stop codon is at a position corresponding to C256 or E249 of SEQ ID NO: 12, in other words a modification corresponding to C256X or E249X, see for example SEQ ID NO:33 and SEQ ID NO:36.

According to a further specific embodiment, the modification comprises fusion to one or more N-terminal and/or C-terminal tags. Specific examples of such tags include but are not limited to fluorescent tags, such as a GFP tag or m-Cherry tag, and/or His-tags.

Specifically, the modified UPO comprises SEQ ID NO:31, SEQ ID NO:32, SEQ ID NO: 33, SEQ ID NO:34, SEQ ID NO:35, or SEQ ID NO:36, or an amino acid sequence having at least 70%, 75%, 80%, 85%, 90%, 95%, 97%, 98%, or 99% identity to SEQ ID NO:31, SEQ ID NO:32, SEQ ID NO:33, SEQ ID NO:34, SEQ ID NO:35, or SEQ ID NO:36.

A further specific embodiment of the invention relates to a modified unspecific peroxygenase (UPO) having increased peroxygenase activity as compared to the unmodified wild-type UPO12, wherein the modified UPO comprises SEQ ID NO:30, or an amino acid sequence having at least 70%, 75%, 80%, 85%, 90% or 95% sequence identity to SEQ ID NO:30. Specifically, said UPO comprises a modification of at least one amino acid corresponding to the amino acid at position S24 of the UPO of SEQ ID NO: 12. Specifically, said modification is an amino acid substitution corresponding to S24F.

According to a specific embodiment, the modified UPO comprising SEQ ID NO: 30, or an amino acid sequence having at least 70% sequence identity to SEQ ID NO: 30, comprises an additional modification of at least one amino acid corresponding to any one or more of amino acids at positions C256, D253, E249, and/or D145 of the UPO of SEQ ID NO:12. More specifically, said UPO further comprises one or more mutations corresponding to C256S, C256X, E249X, D253N, D2531, and/or D145Y.

Specifically, the peroxygenase activity of the modified UPO described herein is about 1.0-fold, 1.1-fold, 1.2-fold, 1.3-fold, 1.4-fold, 1.5-fold, 1.6-fold, 1.7-fold, 1.8-fold, 1.9-fold, or 2.0-fold or more increased when measured in an ABTS assay and/or in a DMP assay as described herein.

One embodiment of the invention relates to an isolated polypeptide having peroxygenase activity, wherein the polypeptide comprises SEQ ID NO:37 (POX27 or UPO27), SEQ ID NO:38 (POX30 or UPO30), SEQ ID NO:39 (POX32 or UPO32), SEQ ID NO: 40 (POX34 or UPO34) or SEQ ID NO:41 (POX39 or UPO39), or an amino acid sequence having at least 70%, 75%, 80%, 85%, 90%, 95%, 97%, 98%, or 99% identity to comprises SEQ ID NO:37 (POX27), SEQ ID NO:38 (POX30), SEQ ID NO:39 (POX32), SEQ ID NO:40 (POX34) or SEQ ID NO:41 (POX39).

Further provided herein is the use of the polypeptides having peroxidase activity described herein as peroxygenase, specifically they are used in a method employing a biocatalyst having peroxygenase activity, which is the polypeptide having peroxidase activity as described herein.

Specifically, the isolated polypeptide having peroxygenase activity, wherein the polypeptide comprises SEQ ID NO:37 (POX27 or UPO27), SEQ ID NO:38 (POX30 or UPO30), SEQ ID NO:39 (POX32 or UPO32), SEQ ID NO:40 (POX34 or UPO34) or SEQ ID NO:41 (POX39 or UPO39), or an amino acid sequence having at least 70%, 75%, 80%, 85%, 90%, 95%, 97%, 98%, or 99% identity to comprises SEQ ID NO:37 (POX27), SEQ ID NO:38 (POX30), SEQ ID NO:39 (POX32), SEQ ID NO:40 (POX34) or SEQ ID NO:41 (POX39) is used as peroxygenase.

Specifically, the polypeptides described herein comprising or consisting of the amino acid sequence of SEQ ID NO:1 (UPO1 mut), SEQ ID NO:2 (UPO2), SEQ ID NO: 4 (UPO4), SEQ ID NO:5 (UPO5), SEQ ID NO:7 (UPO7), SEQ ID NO:11 (UPO11), SEQ ID NO:12 (UPO12), SEQ ID NO: 17 (UPO17), SEQ ID NO: 18 (UPO18), SEQ ID NO: 19 (UPO19), SEQ ID NO: 22 (UPO22), SEQ ID NO:23 (UPO23), SEQ ID NO:24 (UPO24), or SEQ ID NO:25 (UPO25) or the polypeptides described herein comprising an amino acid sequence having at least 70%, 75%, 80%, 85%, 90%, 95%, 97%, 98%, or 99% identity to the polypeptide of SEQ ID NO:1 (UPO1 mut), SEQ ID NO:2 (UPO2), SEQ ID NO:4 (UPO4), SEQ ID NO:5 (UPO5), SEQ ID NO:7 (UPO7), SEQ ID NO:11 (UPO11), SEQ ID NO:12 (UPO12), SEQ ID NO:17 (UPO17), SEQ ID NO:18 (UPO18), SEQ ID NO: 19 (UPO19), SEQ ID NO:22 (UPO22), SEQ ID NO:23 (UPO23), SEQ ID NO: 24 (UPO24), or SEQ ID NO:25 (UPO25) are used as peroxygenase.

Specifically, the newly identified peroxygenase UPO27 (SEQ ID NO:37) has about 72% sequence identity to the peroxygenase UPO12 (SEQ ID NO: 12). Without the signal sequence, UPO27 has about 74% sequence identity to UPO12.

According to a specific embodiment of the invention, the modified unspecific peroxygenases described herein, and the isolated polypeptides having peroxygenase activity described herein are produced according to the method described herein.

### BRIEF DESCRIPTION OF DRAWINGS

FIG. 1: Activity landscapes of 21 different PaDa1 mutant transformants at different pH values. While the mutant samples converted ABTS only really slow at pH 3.5, the conversion was more than 10 times faster at pH 4.5 for several clones.

FIG. 2: Comparison of the slope of absorption of the PaDal mutant of AaeUPO1 variant PaDa1 (UPO1) and UPO 11 both at pH 4.5.

FIG. 3: Comparison of the slope of absorption of the PaDal mutant of AaeUPO1 variant PaDa1 (UPO1) and UPO 12, both at pH 4.5. Also, UPO 12 at pH 5.5 is compared and shown.

FIG. 4: Comparison of the slope of absorption of the PaDal mutant, UPO 17 and UPO 17 without mating factor alpha but with the native signal. All constructs were measured at pH 4.5.

FIG. 5: ABTS peroxidase assay to compare PaDal mutant of AaeUPO1 containing the evolved signal peptide for secretion with the PaDal mutant of AaeUPO1 containing a native signal as well as with the UPOs 7,8, 11, 12 (linked to the short mating factor alpha signal) and UPO17 with its native signal peptide. As a control BSYBG11 was applied on the same microtiter plate. A dark color can be observed for UPO 17, a little less dark for UPO 12 and 11, indicating either high specific peroxidase activity and/or high expression especially for UPO17, but also for other UPOs. The PaDal mutant of AaeUPO1 with evolved signal showed low intensity coloring (indicating low expression in this specific experiment), the PaDal mutant of AaeUPO1 with the native signal sequence showed no coloring that could be observed with the eye. UPOs 7 and 18 as well as empty control strain BSYBG11 also showed no coloring in the peroxidase assay. The assay solution was performed in 200 mM citrate buffer at pH 4.5.

FIG. 6: Diagram of different UPOs converting naphthalene by oxygenation, followed by hydroxy naphthol detection with fast blue, measured photometrically by absorption at 520 nm over 5 minutes.

FIG. 7: Sequence of selected constructs

FIG. 8: Comparison of peroxygenase and peroxidase activities of selected constructs in the Naphthalene-Fast Blue assay and ABTS assay.

FIG. 9: Activity of selected UPO12 variants in relation to wild type UPO12 (clone 1G). Substrates: ABTS, 2,6-DMP, naphthalene; Cultivation: 96 hours in shake flask (48 hours growth/derepression, 48 hours MeOH induction).

FIG. 10: Novel POXs (POX27, POX32, POX34, POX39). Screening results of 8 clones per enzyme. Substrates: ABTS, 2,6-DMP, naphthalene; Cultivation: 96 hours DWP cultivation (48 hours growth/de-repression, 48 hours MeOH induction).

FIG. 11: ABTS-Assay (2.0 mM H₂O₂) results novel POXs (POX27, POX32, POX34, POX39). Eight clones of each variant studied using an 8-fold H2O2 access.

FIG. 12: ClustalW Alignment of wildtype UPO12 and UPO12 variants.

FIG. 13: ClustalW Alignment of newly identified peroxygenase UPO27 (POX27) and wildtype UPO12.

### DESCRIPTION OF EMBODIMENTS

Unless indicated or defined otherwise, all terms used herein have their usual meaning in the art, which will be clear to the skilled person.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

Peroxidases are divided in four superfamilies, the peroxidase catalase superfamily, the peroxidase-cyclooxygenase superfamily, the peroxidase-chlorite dismutase superfamily and the peroxidase-peroxygenase superfamily (Zámocký et al. 2015).

Peroxidases carry iron (III) protoporphyrin IX as prosthetic group and in general catalyze the oxidation of various organic and inorganic compounds and the reduction of peroxides as H₂O₂.

According to Zamocký et al. (2015) peroxidases catalyze four different reactions:

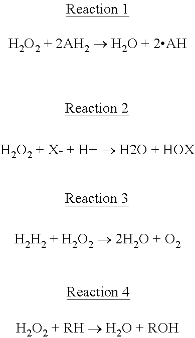

In Reaction 1 electron donors (AH₂) are oxidized to radicals (AH) while H₂O₂ is reduced to water. Reaction 2 shows halides as two-electron donors (X⁻). These are oxidized to hypohalous acids (HOX). The third reaction shows the release of oxygen when a second hydrogen peroxide is used as electron donor. The fourth reaction shows the introduction of oxygen functionalities into organic molecules. Reactions 1 and 2 are common peroxidation reactions, Reaction 3 can be observed only in few heme peroxidases and Reaction 4 is a peroxygenation like reaction, additionally to their peroxidative activity, found in UPOs. The peroxygenase activity reaction mechanism of UPOs is similar to the peroxide shunt pathway of cytochrome P450 enzymes (Zámocký et al. 2015) and bacterial intracellular P450 peroxygenases such as OleT.

According to phylogenetic analyses UPO sequences consist of the motifs (PCP-EGD-R-E) required for the enzyme activity. Both, UPO and CPO have the PCP motif which is required for catalytic activity. The distal cavity of both enzymes consists of a negatively charged glutamic acid residue, which is stabilized by histidine in case of CPO and arginine in case of AaeUPO. In LfuCPO (=CfuCPO) this H105 is involved in the mechanism of its peroxidase function, participating in the cleavage of hydrogen peroxide. The third required motif for catalytic activity in AaeUPO is EGD, which is EHD in CPO. The extended conserved motif for AaeUPO is-PCP-EGD-R-E, and for MroUPO and CPO is-PCP-EHD-E. According to Faiza et al. 2019, most of the putative fungal UPOs reside in Basidiomycota phylum of fungal kingdom. Interestingly MroUPO was placed along with the LfuCPO and some other CPO sequences in the phylogenetic tree. Two new motifs were identified namely, the S [IL] G motif located between the PCP and the EGD motifs and SXXRXD motif present after the EGD motif, except in MroUPO. According to their analysis, all UPOs consist lle in the S [IL] G motif except three species: Jaapia argillacea mucl33604, Mixia osmundae iam14324, and Sphaerulina musiva so2202, which contain Leu in place of lle. This motif was predicted to be relevant for specific substrate selectivity. Thr55 in AaeUPO was predicted to be a critical amino acid residue possibly responsible for driving the functional divergence of UPOs from the CPOs.

Only few wild-type UPOs, including isolated enzymes from Coprinellus radians, Marasmius rotula and A. aegerita have been characterized biochemically. Although more UPOs have been identified based on sequence similarities, these proteins were not isolated and biochemically characterized in detail yet.

So far UPOs were excluded from different possible industrial applications due to missing suitable heterologous expression system. Attempts to functionally express native UPOs in P. pastoris failed or showed nearly undetectable levels of expression (Molina-Espeja et al., 2015) and isolation of such recombinant enzyme from the culture supernatant was not feasible (Molina-Espeja et al. 2015 A wild-type peroxygenase of C. cinera was expressed heterologously in A. oryzae (Babot et al., 2013). In one case expression of stable, soluble AaeUPOs in S. cerevisiae and P. pastoris was brought to an acceptable level through directed evolution over several generations. The activity was measured mainly through ABTS assays with 0.3 mM ABTS and 2 mM hydrogen peroxide (Molina-Espeja et al., 2015).

Further studies showed that there is a similarity of around 30% identity from the sequences of the unspecific peroxygenases AaP and CrP to the sequence of the chloroperoxidase of C. fumago (CfuCPO or LfuCPO). This similarity is located at the N-terminus and comprises the proximal heme-binding region, while the C-terminus is differing completely (Pecyna et al., 2009).

A Blast search of selected sequences of possible unspecific peroxygenases against the sequence of this chloroperoxidase showed similar results with a maximum identity of 25%, but all sequences contained the conserved cysteine residue of the PCP motif that is found in the peroxygenases AaP and CrP as well as in the chloroperoxidase where it serves as fifth heme ligand and has the position Cys29 (Pecyna et al. 2009).

The following alignment, created with Clustal Omega, shows the conserved sequence motifs described above:

| | (SEQ ID NO: 26) | |
|---|---|---|
| 31 | 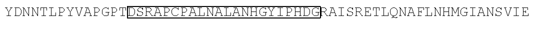 | 90 |
| | (SEQ ID NO: 27) | |
| | 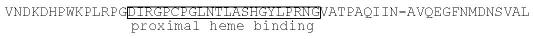 | 118 |
| | (SEQ ID NO: 28) | |
| 91 | 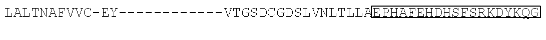 | 138 |
| | (SEQ ID NO: 29) | |
| 119 | 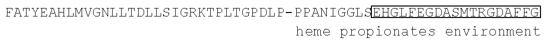 | 177 |

Alignment of the AaP and the CrP peroxygenase with the chloroperoxidase of C. fumago (CfuCPO) demonstrated that the substrate binding is different. Although some epoxidation activity was described for LfuCPO in comparison to UPOs, CPOs are usually not able to epoxidize aromatic rings or to hydroxylate alkanes with the same efficiency.

Thus, it was an object of the invention to evaluate the Pichia system for achieving high yields and titers of new UPOs. The present invention therefore relates to reproducible expression of novel UPOs by the robust and efficient expression system P. pastoris as folded and functional enzymes. The recombinant UPOs of the present invention also showed improved technical properties compared to previously described recombinant UPOs and they can be expressed by secretion by yeast.

The present invention also relates to nucleic acid constructs comprising an isolated polynucleotide of the present invention operably linked to one or more (several) control sequences that direct the expression of the coding sequence in a suitable host cell under conditions compatible with the control sequences. An isolated polynucleotide encoding a polypeptide of the present invention may be manipulated in a variety of ways to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well known in the art. The control sequence may be an appropriate promoter sequence, a nucleotide sequence that is recognized by a host cell for expression of a polynucleotide encoding a polypeptide of the present invention. The promoter sequence contains transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell.

There are several methods to determine the activity of peroxygenases based on different hydrogen donors, such as guaiacol, pyrogallol, ABTS (2,2′-Azino-bis(3-ethylbenzothiazoline-6-sulfonic acid) diammonium salt), 4-methoxyl-α-naphthol and phenol plus amino-antipyrine, or 2,6-DMP (Yuan & Jiang, 2002). Among them, ABTS is a widely used substrate in the spectrophotometric determination of peroxidase and peroxygenase activity because the method is sensitive and the chromogenic products are stable (Putter & Becker, 1983; Yuan & Jiang, 2002).

The peroxygenase activity of the polypeptides having peroxygenase activity described herein, specifically the UPOs described herein, is preferably determined using an ABTS assay or a 2,6-DMP assay.

The ABTS Assay (2,2′-Azino-bis(3-Ethylbenzothiazoline-6-Sulfonic Acid) (ABTS) Enzymatic Assay) is a colorimetric assay based on the ABTS cation radical formation and is well-known in the art, described for example in Pütter & Becker, 1983. The radical formation is catalyzed by the reduction of HRP in the presence of hydrogen peroxide.

According to a specific example, the ABTS assay is carried out analogously as described by Morawski et al. (2000) for horse radish peroxidase (HRP). The ABTS assay may be performed with variable parameters, including varying concentration of the buffer at different pH values. As ABTS assay solution 440 mg 2,2′-azino-bis(3-ethylbenzothiazoline-6-sulfonic acid) in NaOAc may be mixed with buffer and 30% H₂O₂. The cell culture supernatant is mixed with the assay solution and the increase in absorption at 405 nm is measured to determine the peroxidase and/or peroxygenase activity.

The 2,6-DMP assay, or DMP assay in short, is another preferred activity assay that is used to detect and measure peroxygenase activity of the polypeptides described herein. In this method, 2,6-dimethoxyphenol and hydrogen peroxide are used as co-substrates in a nonspecific peroxygenase-catalyzed reaction leading to the formation of a colored product.

To determine an increased activity, a benchmark is also measured in the activity assay. The benchmark may for example be the wild-type polypeptide, not comprising any of the modifications described herein, or the PaDa1 mutant (of AaeUPO1). The benchmark is measured under the same conditions as the polypeptide of interest for which an increased activity shall be determined.

Surprisingly, the modified unspecific peroxygenases described herein comprise an increased peroxygenase activity of about at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5 or 10-fold increased activity as determined by ABTS assay and/or by DMP assay. Surprisingly, the largest group of the UPO12 variants described herein comprising improved peroxygenase activity were found to have a mutation at the C-terminus of the POX12 (UPO12) protein sequence.

The term “C-terminus” (also known as the carboxyl-terminus, carboxy-terminus, C-terminal tail, C-terminal end, or COOH-terminus) as used herein refers to the end of an amino acid chain (protein or polypeptide) comprising a free carboxyl group (—COOH). The C-terminus may comprise any of 5, 10, 15, 20, 25, 50, 100, 150, or 200 amino acids, or any number in between.

Specifically, the term C-terminus as used herein with reference to the modified UPOs described herein, refers to a sequence of amino acids corresponding to amino acids 145-261 of UPO12, preferably amino acids 230 to 261, or even more preferably to the amino acids from position 240 or 250 to 261, of the UPO of SEQ ID NO: 12. Specifically, the sequence corresponding to the C-terminus of SEQ ID NO:12 is not necessarily identical to the C-terminus of SEQ ID NO:12 but shares at least about 70, 75, 80, 85, 90, or 95% sequence identity.

Specifically, the modified UPOs described herein comprise one or more amino acid modifications at positions corresponding to S24, C256, D253, E249, and/or D145 of SEQ ID NO: 12. The position of the amino acid modification may not be identical to positions S24, C256, D253, E249, and/or D145 of SEQ ID NO:12, but it is functionally equivalent to said positions. Identification of functionally equivalent positions is readily available to a person skilled in the art, for example by employing structural alignments.

The polypeptides having peroxygenase activity described herein, specifically the UPOs described herein, including the modified UPOs described herein, can be used in various applications. Specifically, the polypeptides described herein are employed in oxyfunctionalization reactions, oxidative defunctionalization reactions and/or oxidative polymerization reactions. Industrial applications of the UPOs described herein and isolated polypeptides comprising peroxygenase activity are numerous; they reach from pharmaceutical production to environmental applications, including environmental problems caused by industry. For example, transformation of pollutants using the UPOs described herein can result in reduction of toxicity or bioavailability. Also, removal of pollutants from water can be achieved.

The polypeptides having peroxygenase activity described herein, specifically the UPOs described herein, including the modified UPOs described herein, may be further modified, such modifications including for example insertion or deletion of post-translational modification sites, insertion or deletion of targeting signals (e.g.: leader peptides), fusion to tags, linker peptides, proteins or protein fragments facilitating their processing such as purification or detection or enhancing their stability.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, linker peptides causing ribosomal skipping, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” denotes herein a configuration in which a control sequence is placed at an appropriate position relative to the coding sequence of the polynucleotide sequence such that the control sequence directs the expression of the coding sequence of a polypeptide.

The term “expression” includes any step involved in the production of the polypeptide including, but not limited to, transcription, post-transcriptional modification, translation, post-translational modification, and secretion.

The term “expression vector” is defined herein as a linear or circular DNA molecule that comprises a polynucleotide encoding a polypeptide of the present invention and is operably linked to additional nucleotides that provide for its expression.

The term “functional variant” or “functionally active variant” also includes naturally occurring allelic variants, as well as mutants or any other non-naturally occurring variants of the UPOs described herein. As is known in the art, an allelic variant is an alternate form of a nucleic acid or peptide that is characterized as having a substitution, deletion, or addition of one or nucleotides or more amino acids that does essentially not alter the biological function of the nucleic acid or polypeptide.

Functional variants may be obtained by sequence alterations in the polypeptide or the nucleotide sequence, e.g. by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

A point mutation is particularly understood as the engineering of a poly-nucleotide that results in the expression of an amino acid sequence that differs from the non-engineered amino acid sequence in the substitution or exchange, deletion or 5 insertion of one or more single (non-consecutive) or doublets of amino acids for different amino acids.

The term “heterologous” as used herein with respect to a nucleotide or amino acid sequence or protein, specifically the UPOs and promoters described herein, refers to a compound which is foreign, i.e. “exogenous”, such as not found in nature, to a given host cell. The heterologous nucleotide sequence may also be expressed in an unnatural, e.g., greater than expected or greater than naturally found, amount in the cell. Specifically, heterologous nucleotide sequences are those not found in the same relationship to a host cell in nature (i.e., “not natively associated”). Any recombinant or artificial nucleotide sequence is understood to be heterologous. An example of a heterologous polynucleotide or nucleic acid molecule comprises a nucleotide sequence not natively associated with a promoter, e.g., to obtain a hybrid promoter, or operably linked to a coding sequence, as described herein. As a result, a hybrid or chimeric polynucleotide may be obtained. A further example of a heterologous compound is a UPO-encoding polynucleotide or gene operably linked to a transcriptional control element, e.g., a promoter, to which an endogenous, naturally-occurring POI coding sequence is not normally operably linked.

“Sequence identity” as described herein is defined as the percentage of nucleotides or amino acid residues in a candidate sequence that are identical with the nucleotides or amino acid residues in the specific nucleotide or polypeptide sequence to be compared (the “parent sequence”), after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

The term “operably linked” as used herein refers to the association of nucleotide sequences on a single nucleic acid molecule, e.g. the vector, plasmid or chromosome, in a way such that the function of one or more nucleotide sequences is affected by at least one other nucleotide sequence present on said nucleic acid molecule. For example, a promoter is operably linked with a coding sequence encoding a UPO described herein, when it is capable of effecting the expression of that coding sequence. Specifically, such nucleic acids operably linked to each other may be immediately linked, i.e. without further elements or nucleic acid sequences in between or may be indirectly linked with spacer sequences or other sequences in between.

The term “host cell”, as used herein, includes any cell type that is susceptible to transformation, transfection, transduction, and the like with a nucleic acid construct or expression vector comprising a polynucleotide of the present invention.

Specifically, the host yeast cells are maintained under conditions allowing expression and/or secretion of the peroxygenases described herein.

In one aspect the host cell is a yeast cell. “Yeast” as used herein includes ascosporogenous yeast (Endomycetales), basidiosporogenous yeast, and yeast belonging to the Fungi Imperfecti (Blastomycetes). In one aspect, the yeast host cell is a Candida, Hansenula, Kluyveromyces, Pichia, Saccharomyces, chizosaccharomyces, or Yarrowia cell. In a further aspect, the yeast host cell is a Pichia pastoris cell.

Specifically, the methylotrophic yeasts Komagataella (Pichia) pastoris, Komagataella (Pichia) phaffii (Pp), Komagataella Kurtzmanii, Ogataea (Hansenula) polymorpha (Hp), Candida boidinii (Cb) and Ogataea (Pichia) methanolica (Pm) have been established as efficient alternative production strains. These strains make it possible to achieve high expression rates for heterologous proteins with a high cell density. Of the aforementioned four yeast species, P. pastoris (Komagataella phaffii) has in the meantime been used most commonly for heterologous protein production.

The term “methylotrophic yeast cells”, as used herein, includes yeast cells capable of growing on culture media containing as carbon source substances with only one carbon atom, for example methanol.

The term “promoter” as used herein refers to an expression control element that permits binding of RNA or DNA polymerase and the initiation of transcription.

“Derepressing conditions”, as used in culturing the yeast cells according to one aspect, means that the yeast cells are first cultured in the presence of a repressing carbon source (e.g. glucose) until this carbon source has been mostly or entirely consumed. After reducing the concentration of the repressing carbon source (e.g. glucose), the cells are in derepressing conditions with respect to the repressing carbon source and glucose, respectively. The strength of the repression effects may depend on the type of carbon source and on specific growth rates.

Derepressed promoter sequences are activated by de-repression upon carbon source limitation and depletion and not upon induction by methanol.

The derepressed and methanol-independent promoters used according to the present invention display at least 10% activity in a suitable environment that does not comprise methanol. Preferably, such promoters comprise at least 10%, 15%, 20%, 25%, 30%, 35%, 40%, 45%, 50%, 55%, 60%, 65%, or 70% under derepressing conditions and without the addition of methanol.

In contrast, methanol-dependent promoter sequences, such as the AOX1 promoter, display less than 1% activity, typically less than 0.1% or even less, without the addition of methanol to the cell culture.

In a yeast host, useful promoters are for example, AOX1, PDC, and PDF, FMD and FDH or FLD promoters and peroxisomal catalase gene promoters of different methylotrophic yeast as well as for example promoters of genes, coding for peroxisomal proteins. According to a preferred embodiment, the PDC or FMD promoter is used in the method described herein.

The term “signal peptide”, as used herein, refers to a peptide linked to the C-terminus or N-terminus of the polypeptide, which controls the secretion of the polypeptide. The signal sequence used may be a polynucleotide which codes for an amino acid sequence which initiates the transport of a protein through the membrane of the endoplasmic reticulum (ER). The nucleic acid sequence of these signal sequences may correspond to the natural sequence of the original host cell or may be codon-optimized. The non-limited examples of the signal sequence include native fungal plant or animal protein signal sequences, MF-alpha (“mating factor alpha” signal sequence), the OST1 signal peptide, the signal sequence of the CBH2 protein from Trichoderma reesei, the signal sequence of the xylanase A from Thermomyces lanuginosus, KI killer toxin signal, the signal peptide for invertase secretion, the signal sequence of the killer toxin from Kluyveromyces lactis, the signal sequence of the killer toxin from Pichia acaciae, the signal sequence of the killer toxin from Hanseniaspora uvarum and from Pichia (Hansenula) anomala or variants thereof and signal sequences of proteins exposed at the surface of P. pastoris. In one aspect, the preferred signal sequence is MF-alpha (“mating factor alpha” signal sequence). According to a further preferred aspect, the signal sequence is a signal sequence from Podospora anserine.

A suitable expression system is for example disclosed in WO2017/109082.

On aspects related to the selection and codon optimization of sequences, expression system and confirmation of the activity of the enzymes. Different new enzyme sequences showing a clear difference to so far known enzymes were identified and provided.

The term “cell culture” or “cultivation” (“culturing” is herein synonymously used), also termed “fermentation”, with respect to a host cell line is meant to be the maintenance of yeast cells in an artificial, e.g., an in vitro environment, under conditions favoring growth, differentiation or continued viability, in an active or quiescent state, of the cells, specifically in a controlled bioreactor according to methods known in the industry. When cultivating, a cell culture is brought into contact with the cell culture media in a culture vessel or with substrate under conditions suitable to support cultivation of the cell culture and expression and/or secretion of the peroxygenases described herein. Specifically, a culture medium is used to culture cells according to standard cell culture techniques that are well-known in the art for cultivating or growing yeast cells.

Cell culture may be a batch process or a fed-batch process. A batch process is a cultivation mode in which all the nutrients necessary for cultivation of the cells, and optionally including the substrates necessary for production of the carbonyl compounds described herein, are contained in the initial culture medium, without additional supply of further nutrients during fermentation. In a fed-batch process, a feeding phase takes place after the batch phase. In the feeding phase one or more nutrients, such as the substrate described herein, are supplied to the culture by feeding. In certain embodiments, the method described herein is a fed-batch process. Specifically, a host cell transformed with a nucleic acid construct encoding the polypeptides described herein, specifically the UPOs as described herein, is cultured in a growth phase medium and transitioned to an induction phase medium in order to produce the polypeptides described herein.

In another embodiment, host cells described herein are cultivated in continuous mode, e.g. a chemostat. A continuous fermentation process is characterized by a defined, constant and continuous rate of feeding of fresh culture medium into the bioreactor, whereby culture broth is at the same time removed from the bioreactor at the same defined, constant and continuous removal rate. By keeping culture medium, feeding rate and removal rate at the same constant level, the cultivation parameters and conditions in the bioreactor remain constant.

Suitable cultivation techniques may encompass cultivation in a bioreactor starting with a batch phase, followed by a short exponential fed batch phase at high specific growth rate, further followed by a fed batch phase at a low specific growth rate. Another suitable cultivation technique may encompass a batch phase followed by a continuous cultivation phase at a low dilution rate.

It is preferred to cultivate the host cell line as described herein in a bioreactor under growth conditions to obtain a cell density of at least about 1 g/L, 5 g/L or 10 g/L cell dry weight, more preferably at least 20 g/L cell dry weight, preferably at least 50 g/L cell dry weight. It is advantageous to provide for such yields of biomass production on a pilot or industrial scale.

The term “mutation” as used herein has its ordinary meaning in the art. A mutation may comprise a point mutation, or refer to areas of sequences, in particular changing contiguous or non-contiguous amino acid sequences. Specifically, a mutation is a point mutation, which is herein understood as a mutation to alter one or more (but only a few) contiguous amino acids, e.g. 1, or 2, or 3 amino acids, which are substituted, inserted or deleted at one position in an amino acid sequence. Amino acid substitutions may be conservative amino acid substitutions or non-conservative amino acid substitutions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a poly-nucleotide that results in the expression of an amino acid sequence that differs from the non-engineered amino acid sequence in the substitution or exchange, deletion or insertion of one or more single (non-consecutive) or doublets of amino acids for different amino acids.

The term “functional variant” or “functionally active variant” also includes naturally occurring allelic variants, as well as mutants or any other non-naturally occurring variants. As is known in the art, an allelic variant is an alternate form of a nucleic acid or peptide that is characterized as having a substitution, deletion, or addition of one or nucleotides or more amino acids that does essentially not alter the biological function of the nucleic acid or polypeptide. Functional variants may be obtained by sequence alterations in the polypeptide or the nucleotide sequence, e.g. by one or more point mutations, wherein the sequence alterations retain or improve a function of the unaltered polypeptide or the nucleotide sequence, when used in combination of the invention. Such sequence alterations can include, but are not limited to, (conservative) substitutions, additions, deletions, mutations and insertions.

In one aspect as described herein, several UPOs from basidiomycetes and ascomycetes were identified and studied. In Table 1 the constructs that have been tested are listed with their associated accession numbers.

TABLE 1
Tested UPO and CPO candidates

| Candidate | Accession number | Notes |
|---|---|---|
| UPO 1mut | B9W4V6 | PaDa I mutant |
| UPO 2 | KDR72024.1 | Unspecific peroxygenase |
| UPO 3 | KJA13294.1 | Unspecific peroxygenase |
| UPO 4 | XP_006458802 | Unspecific peroxygenase |
| UPO 5 | KIK06072.1 | Unspecific peroxygenase |
| UPO 6 | KIJ31387.1 | Unspecific peroxygenase |
| UPO 7 | KIM43689.1 | Unspecific peroxygenase |
| UPO 8 | KJA24696.1 | Unspecific peroxygenase |
| UPO 9 | ESZ93716.1 | Unspecific peroxygenase |
| UPO 10 | CAK39169.1 | Unspecific peroxygenase |
| UPO 11 | OJJ73116.1 | Unspecific peroxygenase |
| UPO 12 | OTA57433.1 | Unspecific peroxygenase |
| UPO 13 | XP_001225194.1 | Unspecific peroxygenase |
| UPO 14 | XP_001219540.1 | Unspecific peroxygenase |
| UPO 15 | KIJ30163.1 | Unspecific peroxygenase |
| UPO 16 | KIJ46203.1 | Unspecific peroxygenase |
| UPO 17 | XP_001911526.1 | Unspecific peroxygenase |
| UPO 18 | XP_006459044.1 | Unspecific peroxygenase |
| CPO 19 | CAA28172 | Chloroperoxidase |
| CPO 20 | AJA36817 | Chloroperoxidase |
| UPO21* | CAV28569.1 | Unspecific peroxygenase |
| UPO22 | OTB17553.1 | Unspecific peroxygenase |
| UPO23 | GAQ45152.1 | Unspecific peroxygenase |
| UPO24 | XP_001390900.2 | Unspecific peroxygenase |
| UPO25 | GAA88053.1 | Unspecific peroxygenase |

In one aspect, the polypeptide comprises an amino acid sequence having at least 70%, 75%, 80%, 85%, 90%, 95%, 97%, 98%, or 99% identity to the polypeptide of SEQ ID NO:11 (UPO11).

In one aspect, the polypeptide comprises an amino acid sequence having at least 70%, 75%, 80%, 85%, 90%, 95%, 97%, 98%, or 99% identity to the polypeptide of SEQ ID NO: 12 (UPO12).

In one aspect, the polypeptide comprises an amino acid sequence having at least 80%, 85%, 90%, 95%, 97%, 98% or 99% identity to the polypeptide of SEQ ID NO: 17 (UPO17).

In one aspect, the polypeptide comprises an amino acid sequence having at least 95% identity to the polypeptide of and SEQ ID NO:23 (UPO23).

In one embodiment as described herein, using Pichia pastoris as expression system with a methanol-independent PDC promoter and the engineered gene/protein sequence as described herein more than 200 mg/L secreted enzyme were obtained. One aspect provides for yields of 0.5 g/L or even 1 g/L of the desired enzyme. This yield came close to secreted UPO concentrations observed in native hosts.

### EXAMPLES

The Examples which follow are set forth to aid in the understanding of the invention but are not intended to, and should not be construed to limit the scope of the invention in any way. The Examples do not include detailed descriptions of conventional methods, e.g., cloning, transfection, and basic aspects of methods for expressing proteins in microbial host cells. Such methods are well known to those of ordinary skill in the art.

### Materials and Methods

### Sequence Selection Procedure

Sequences described in databases were analyzed or potential peroxygenase activity using various free available sequence databases, e.g. genbank at NCBI with the data sets nonredundant or patdb

Google Patent search, Canadian Patents Database, Patentscope, Espacenet, and DPMA.

Searches were done based by blast searches using previously published sequences with known or claimed activities as input.

Signal BLAST and SignalP were used for analyzing all those sequences individually in order to find out if a hypothetical protein is potentially secreted and to identify predictable signal sequence cleavage sites, enabling the replacement of native signal peptides by others such as the signal sequence of the S. cerevisiae mating factor alpha.

A Multiple Sequence Alignment as well as a phylogenetic tree were obtained by Clustal Omega analysis, which uses the neighbor-joining method for the phylogenetic tree.

Out of this huge amount of data a matrix for choosing sequences was generated. Two big groups were identified depending on the similarity, with each of them having several hotspots containing notably high similarities as it showed up, in the heatmap, which supported the final decision process.

Final sequence selections were made based on the sequence comparisons in order to stay in distance from previously known and/or characterized UPOs, for reflecting a broad coverage of sequence diversity in the phylogenetic tree and to cover a broad sequence diversity which also might reflect functional diversity. However also known heme thiolate peroxygenases such as the AaeUPO1 variant PaDa1 and the CfuCPO were included in the performed expression studies. The evolved AaeUPO1 variant served as a positive control for expression and activity tests, while the CPO was used as one of the negative controls, since functional secretory expression by Pichia pastoris was reported to have failed in other labs before.

### Sequencing Genes of Interest

Single colonies that (due to the colony PCR) were likely to contain the cloned peroxygenase reading frames, cloned into the expression vector, inter alia, were streaked out on LB Zeocin plates and incubated at 37° C. overnight to amplify the recombinant plasmid.

A minipreparation of plasmid DNA was done with Promega's “Wizard® Plus SV Minipreps DNA Purification System” as described herein.

For sequence verification and analysis of potential errors produced by DNA synthesis or PCR amplification/cloning, isolated plasmid DNA was sent for Sanger sequencing of the DNA. Therefore at least 1,200 ng of DNA plus 3 μL of 10 μM forward or reverse primer, respectively, were brought to a total volume of 15 μL with dH₂O.

P. pastoris Transformation

For the transformation of electrocompetent cells and for genomic integration of the expression cassettes the vectors were linearized by a single cut using Swal. Deviating from standard protocols only 0.5 μL of enzyme were used and the incubation time was increased to three hours after checking in the enzyme manufacturers description that the used restriction enzyme has no star activity.

The linearized expression cassettes were desalted by dialysis using filter discs floating on water, before the DNA was used for Pichia transformation.

For one transformation 40 μL of ready to use electrocompetent Pichia pastoris BSYBG11 cells (Table 6, Bisy GmbH, Austria) and around 1 μg of linearized plasmid DNA were used.

First of all the competent cells were defrosted on ice and the cuvettes were cooled. Then the competent cells and the plasmid DNA were pooled in the cooled cuvettes and kept on ice for at least 10 minutes. Afterwards electroporation was carried out with a voltage of 1.5 kV, followed by the addition of regeneration medium (YPD/1 M Sorbitol, 1:1 (v/v)).

The mixture containing transformants and regeneration medium was transferred to Eppendorf tubes (0) and regenerated for 2 hours at 30° C. and 700 rpm, followed by a centrifugation step (1 min, full speed). The supernatant was reduced to 100 μL, the cell pellet was resuspended therein and plated on LB-Zeocin plates. The plates were incubated at 30° C. for two days.

### Cultivation

Cultivation was carried out in deep well plates either as one or as two-day(s) induction.

### Two-Day Induction:

Single colonies of the transformed Pichia pastoris cells (from 0) were picked with sterile toothpicks. Then they were transferred to the wells of the deep-well-plates, containing 300 UL BMD1 per well, and incubated at 28° C. with 320 rpm for 36-60 hours.

After this incubation they were induced by methanol adding 250 μL of BMM2 per well and incubated again. 12, 24 and 36 hours later 50 UL of BMM10 were added per well.

12 hours after the last addition of BMM10 the deep-well plates were centrifuged for 10 minutes at high speed. The supernatant containing the secreted enzyme was used for the assays described herein.

### One Day Induction

The one-day induction protocol was following the same procedure as the two-day induction protocol but the cells were only induced for the first two times, followed by the harvest already on the next day.

### Flask Cultivation

The Flask cultivation was carried out as follows:

450 mL of BMD1% were inoculated with the transformed Pichia pastoris as described above in a 2.5 L Ultra Yield Flask (UYF).

The flasks were incubated for 3 days at 28° C. and 100 rpm.

After incubation the induction was started with 50 mL of BMM1). Every 12 hours 5 mL of 100% methanol were added for three times.

The day after the last induction the culture was harvested by centrifugation in 500 mL tubes for 15 minutes at 8,000 rpm. The enzyme was in the supernatant. Cells were removed by centrifugation. The supernatants were filtered through a membrane with a pore size of 0.45 μm and stored at 4° C.

The concentration of the enzyme in the supernatant was evaluated by centrifugation with Vivaspin columns with a 10 kD cutoff.

### Bioreactor Cultivation

To scale up enzyme production Sartorius 5 L bioreactors were used for cultivations.

The bioreactor cultivations were based on Invitrogen's™ “Pichia Fermentation Process Guidelines”. In detail the cultivation was done as follows:

The pre-culture I, consisting of 50 mL BMGY in 250 ml baffled flask with some cell material of transformants grown on an agar plate, was incubated at 110 rpm, 28° C. and about 50% humidity for about 60 hours.

After the incubation an aliquot of preculture I was used to inoculate the pre-culture II (200 mL BMGY in 1 L baffled flask) to an OD600 of 3.0. After about four hours the 3.5 L BSM medium in the 5 L bioreactor was inoculated to an OD600 of about 1.0 (as measured with the same photometer). The glycerol batch phase lasted for 22 hours until the entire carbon source was consumed.

The standard conditions in a non-optimized bioreactor cultivation was: 28° C., pH 6.0, min. stirring at 500 rpm, min. dO2 of 30% (cascade setting) and 4 L/min airflow.

During the glycerol fed-batch phase the culture was fed constantly with 26 mL/h/L (L . . . liter of start volume; 3.5 L: 91 mL/h) 50% glycerol with PTM1 and biotin (both 12 mL/L fed-batch medium) for 6 hours. During night the culture was fed with 2.6 mL/h/L fed-batch medium.

On the next morning the glycerol-feed was turned off and after 30 minutes 100% methanol was added to the bioreactor culture to a final methanol concentration of 1%. After consumption a constant methanol feed was set to 3 mL/h/L (L . . . liter of start volume; 3.5 L: 10.5 mL/h) pure methanol (without PTM1 or biotin). This flow rate was kept for 30 hours.

Finally, the culture was harvested in 1 L centrifuge tubes at 8,000 rpm and the supernatant was transferred into clean bottles and stored at 4° C. until future use.

### Activity Assays

Well described standard assays for measuring the activity of unspecific peroxygenases were applied for proving the peroxidase and/or peroxygenase activities of the supernatants of the performed cultivations.

### ABTS Assay

The ABTS assay was carried out analogously as described by Morawski et al. (2000) for horse radish peroxidase (HRP). The ABTS assay was performed with variable parameters, including varying concentration of the buffer at different pH values.

TABLE 2
Assay Solution

| Variable parameters | Range used | |
|---|---|---|
| supernatant | 2-15 | μL |
| buffer | NaOAc or citrate-phosphate-buffer | |
| pH value of buffer | 2.5-7.0 | |
| molarity of buffer | 100-200 | mM |

For one 96-well plate 20 mL assay solution were prepared. Therefore 1 mL 20× ABTS stock solution (440 mg 2,2′-azino-bis(3-ethylbenzothiazoline-6-sulfonic acid) in 50 mL 50 mM NaOAc) was mixed with 19 mL buffer and 1.75 μL 30% H₂O₂. The assay solution was kept on ice.

15 μL of the supernatant was mixed with 140 μL of the assay solution and the increase in absorption at 405 nm was measured with the plate reader.

The first screening was carried out with a buffer concentration of 50 mM and pH values of 3.5, 4.5 and 5.5 respectively. As benchmark the PaDa1 mutant (of AaeUPO1) was measured under the same conditions as the new constructs. The new UPO constructs were measured after secretion employing their natural signal peptide as well as the alternative mating factor alpha signal peptide. The measurement was carried out for 15 minutes.

### 2,6-DMP Assay

The 2,6-DMP (2,6-dimethoxyphenol) assay was done similar to the assay described by E. Breslmayr et al. (2018) for lytic polysaccharide monooxygenases and by P. Molina-Espeja et al. (2016). The 2,6-DMP assay was performed with potassium phosphate (KPi) buffer pH 7.0.

For one 96-well plate 20 mL assay solution were prepared. Therefore 2 mL 2,6-DMP stock solution (100 mM, 154 mg in 10 mL ddH₂O, heated to 60° C. for better solubility) was mixed with 2 mL KPi buffer (1.0 M, pH 7.0), 16 mL ddH₂O and 0.5 μL 33% H₂O₂. The assay solution was kept on ice.

15 μL of the supernatant was mixed with 185 μL of the assay solution and the increase in absorption at 469 nm was measured with the plate reader. The measurement was carried out for 9 minutes.

### Naphthalene Assay

This assay for aromatic peroxygenases was done similar to the Naphthalene-Fast Blue-Assay described by Gröbe et al. (2011). For one 96-well plate 20 ml of assay solution were prepared.

TABLE 3
Assay Solution

| Variable parameters | Range used | |
|---|---|---|
| supernatant | 2-15 | μL |
| buffer | NaOAc or citrate-phosphate- | |
| | buffer | |
| pH value of buffer | 4.5-5.5 | |
| molarity of buffer | 100-200 | mM |
| Naphthalene stock solution | 1-2 | mL |
| Fast Blue stock solution | 1-2 | mL |

4 mM naphthalene stock solution: 5 mg naphthalene, in 10 mL acetone

2 mM Fast Blue stock solution: 9.5 mg Fast Blue B salt/10 mL dH₂O

For one 96-well plate 20 mL assay solution were prepared, containing 10 mL citrate-phosphate-buffer, 1-2 mL 4 mM naphthalene stock solution and the same amount of 2 mM Fast Blue stock, 2 μL 30% H₂O₂. Then dH₂O was added to obtain a final volume of 20 mL.

30 μL of the supernatant was mixed with 150 μL of assay solution and the increase in absorption at 520 nm was measured with plate reader.

### Filter Assay

The filter assay was done similar to the ABTS assay described above. For proof of concept, HRP secreting Pichia pastoris BSYBG11 strains were used as positive control and wild type BSYBG11 strains as negative control.

The positive and negative controls were streaked out on agar plates containing Zeocin to get single colonies. The plates also contained methanol for induction. A filter paper was laid on the plate, so that the colonies stick to it. This filter was transferred to an empty petri dish with the colonies looking down. This should help to keep the colonies where they are, not washing them off. Then 100 μL of assay solution, as described above, where carefully pipetted onto the filter. The assay was incubated at room temperature and controlled every five minutes.

After the concept was proven by seeing green color development due to ABTS oxidation on agar plates by HRP producing cells, the same assay was conducted with UPO 1, 12 and 17 secreting P. pastoris BSYBG11 as positive control and wild type P. pastoris BSYBG11 as negative control.

### Plate Assay

It was assumed that a colony secreting an active UPO would be surrounded by a greenish halo, similar to the filter paper assay. To have a proof of concept the plate assay was carried out with an HRP (horse radish peroxidase) secreting P. pastoris BSYBG11 transformant as positive control and a P. pastoris BSYBG11 wild type as negative control.

The plates where made with buffered minimal medium containing 1% of methanol, sorbitol or glucose respectively. H₂O₂, 30% (Table 5) was added to final concentrations of 43.3 μL*L-1, 87.5 μL*L-1 and 175 μL*L-1, respectively.

The positive and negative controls were streaked out and the plates where incubated at 28° C. for two days. After that time single colonies should be formed. The plates were evaluated by visual inspection.

### Volumetric Peroxidase Activity

Peroxidase activity measurements were performed in plate readers, normalizing by respective assay volumes in the plates. For the calculation of units, the layer thickness was calculated according to Formula 1. In Formula 1 the “h” value corresponds to the layer thickness “d”.

Formula 1: The layer thickness was calculated depending on the total volume per well.

$$π * [ ( {h}^{3} * {tan}^{2} ϕ ) / 3 * h \hat{} 2 * r * tan ϕ + h * {r}^{2} ] - V = 0$$

After determining the layer thickness, the units were calculated with Formula 2.

Formula 2: Calculation of volumetric peroxidase activity.

$$U = ( Δ A Δ {t}^{- 1} * {V}_{tot} * D ) / ( {v}_{sample} * {ε}_{405} * d )$$

U Units per mL [μmol*ml−1*min−1]
Vtot total assay volume [mL]
ΔAΔt⁻¹ change in absorption per time [ΔA(405)*min⁻¹]
D dilution factor of the sample
d layer thickness [cm]
vsample sample volume [mL]
ε405 extinction coefficient at 405 nm [36,000 mL*μmol−1*cm−1]

### Bioconversions

As there are many known substrates that are converted by UPOs just a few exemplifying substrates were tested to proof that the new UPOs are active and able to convert those model substrates. To verify possible bio-conversions HPLC measurements were carried out.

To be able to carry out HPLC measurement including control samples also supernatants from P. pastoris BSYBG11 cultures, grown as negative control and even pure substrate in assay buffer (without enzymes) were applied on the 96-well plate. Furthermore, transformed strains expressing two other intracellular enzymes, human Cytochrome P4502C9 and 3A4, were used as benchmark and control.

The following substrates of interest have been tested: Chlorzoxazone, testosterone, clopidogrel, diclofenac, dextromethorphan, estriol, ethionamide, ibuprofen, lidocaine, and moclobemide.

Bioconversions were carried out in 96-well deep well plates. The assay buffer consisted of 20 mL of 200 mM citrate-phosphate buffer at pH 4.7 containing 2 μL of 30% (w/w) H₂O₂.

The refreshing buffer consisted of 20 ml of 200 mM citrate-phosphate buffer at pH 4.7 containing 200 μL of 30% (w/w) H₂O₂.

Each well contained 100 UL of supernatant, 100 UL of assay buffer and 4 μL of stock substrate solution (100 mM). The deep well plates were incubated at 28° C. and 320 rpm for 15 hours. 0.5 μL of refreshing buffer were added per well. The deep well plates were re-incubated for another 6 hours. To stop the conversion 150 UL of an acetonitrile/methanol (1:1) mixture was added.

For sample preparation, the polypropylene microtiter plates were centrifuged for 20 minutes at 4000 rpm and 4° C. 100 μL of reaction supernatant was transferred into a fresh polypropylene microtiter plate. The new plate was used for measurements by HPLC.

The applied HPLC parameters are listed in Table 4.

The analyses were done on an Agilent 1200 series HPLC system (Agilent technologies, Santa Clara, California, USA) coupled with a mass spectrometer detector (MSD) containing an electron spray ionization unit.

TABLE 4
HPLC-MS parameters

| | | Column |
|---|---|---|
| | | (Kinetex ® 2.6 μm C18 100 Å, LC |
| Parameters | | Column 50 × 4.6 mm) |
| LC | Injection volume (μL) | 10 |
| | Flow rate (ml min⁻¹) | 1 |
| | Column temp. (° C.) | 25 |
| | Solvent A1 | H₂O-0.1% acetic acid |
| | Solvent B2 | ACN |
| API-ES | Gas temp. (° C.) | 350 |
| | Gas flow (L min⁻¹) | 12 |
| | Nebulizer (psi) | 35 |
| | Quad temp. (° C.) | 350 |
| | Polarity | Positive |

### Materials

### Chemicals

TABLE 5
List of used chemicals.

| Name | Provider |
|---|---|
| LB-Medium | Carl Roth, GmbH, Karlsruhe, Germany |
| l0x FastDigest Buffer | Thermo Scientific Inc., Massachusetts, |
| | USA |
| 5x Q5 ® Reaction Buffer | New England Biolabs, Ipswich, MA, |
| | United States |
| ABTS | Sigma-Aldrich Chemie GmbH, Missouri, |
| | USA |
| Agar-Agar | Carl Roth, GmbH, Karlsruhe, Germany |
| BD Bacto ™ yeast extract | Becton, Dickinson and Company, Sparks, |
| | MD, USA |
| Biotin | Sigma-Aldrich Chemie GmbH, Missouri, |
| | USA |
| Biozym LE agarose | Biozym Scientific, Hessisch Oldendorf, |
| | Germany |
| dATP, dGTP (10 mM) | Thermo Scientific Inc., Massachusetts, |
| | USA |
| dCTP, dTTP (20 mM) | Thermo Scientific Inc., Massachusetts, |
| | USA |
| D-Glucose-monohydrate | Carl Roth, GmbH, Karlsruhe, Germany |
| dNTPs | Thermo Scientific Inc., Massachusetts, |
| | USA |
| D-Sorbit | Carl Roth, GmbH, Karlsruhe, Germany |
| FastBlue B | Sigma-Aldrich Chemie GmbH, Missouri, |
| | USA |
| FastDigest Green Buffer | Thermo Scientific Inc., Massachusetts, |
| | USA |
| Hydrogen peroxide | Carl Roth, GmbH, Karlsruhe, Germany |
| K₂HPO₄ | Carl Roth, GmbH, Karlsruhe, Germany |
| KH₂PO₄ | Carl Roth, GmbH, Karlsruhe, Germany |
| Methanol | Sigma-Aldrich Chemie GmbH, Missouri, |
| | USA |
| MgCl₂ (25 mM) | Thermo Scientific Inc., Massachusetts, |
| | USA |
| Naphthalene | Sigma-Aldrich Chemie GmbH, Missouri, |
| | USA |
| Trichloroacetic acid (TCA) | Sigma-Aldrich Chemie GmbH, Missouri, |
| | USA |

### Host Strains

For the transformation with linearized integrative plasmid DNA vectors containing putative new UPO gene sequences the Pichia pastoris platform strain BSYBG11 was used. Compared to the wild type strain BSYBg10, this strain has an AOX1 gene knock out leading to a slow growth phenotype, when methanol is used as carbon source.

TABLE 6
Information on the origin strains used to
generate Pichia pastoris biocatalysts.

| | Species/Host strain: | |
|---|---|---|
| | Komagataella phaffii | Komagataella phaffii |
| | BSYBG10 | BSYBG11 |
| | (Syn.: Pichia pastoris) | (Syn.: Pichia pastoris) |
| BT Culture collection | 7287 | 7288 |
| number of IMBT at TU | | |
| Graz | | |

### Media, Buffers and Solutions

Media used during the thesis are conventional media. If not mentioned else the amounts are given for 1 L of media and the media is autoclaved.

### Plasmids

Plasmids were kindly provided by Bisy GmbH (Austria) and are listed in Table 7.

TABLE 7
Plasmids.

| Plasmid | Promoter | Additional Information |
|---|---|---|
| pBSY3Z | PDC | Contains P. pastoris is CTA1 promoter |
| pBSY3S1Z | PDC | contains additional coding region for short |
| | | variant of mating factor alpha signal |
| pBSY5Z | PDF | Contains Hansenula polymorpha FMD |
| | | promoter variant |
| PBSY5S1Z | PDF | contains additional coding region for short |
| | | variant of mating factor alpha signal |

### Results and Discussion

### Evaluation of Synthetic Heme Thiolate Peroxygenase Genes

After vector digestion with Lgul (Sapl) the stuffer of the vectors pBSY3Z and pBSY3S1Z was cut out. For the vector pBSY3Z also a control digest was performed with EcoRI. Inserts coding for the peroxygenases were inserted into the vector backbones by recombination cloning and transformation of E. coli by electroporation. After plasmid isolation sequences were evaluated by Sanger sequencing. Table 8 shows the results of the sequence evaluation of cloned UPO and CPO genes synthesized by TWIST. In total 24 genes were sequenced, 19 of them proved to be correct. This corresponds to a validity of 79.17%.

The table 8 shows how many genes of each ordered construct were sequenced and how many of them where confirmed.

TABLE 8
Evaluation of synthetic genes.

| Ordered Gene | Correct genes | Genes sequenced |
|---|---|---|
| UPO 2 | 1 | 1 |
| UPO 4 | 1 | 1 |
| UPO 5 | 1 | 3 |
| UPO 6 | 2 | 2 |
| UPO 7 | 2 | 2 |
| UPO 8 | 1 | 2 |
| UPO 9 | 2 | 3 |
| UPO 10 | 2 | 2 |
| UPO 11 | 1 | 2 |
| UPO 13 | 1 | 1 |
| UPO 14 | 1 | 1 |
| UPO 16 | 1 | 1 |
| UPO 17 | 1 | 1 |
| UPO 18 | 1 | 1 |
| CPO 20 | 1 | 1 |
| Total | 19 | 24 |

The pairwise alignments show the identity of the selected new putative UPO candidates to previously known sequences. The alignment was made with Clustal Omega using the full available sequence length. The identity shows the percentage of amino acid sequence identity as given by the “percent identity matrix” created by clustal2.1. An overview of the identities can be found in Table 9 and Table 10.

Due to very low sequence identity the analysis performed with the CPO sequence (CPO19 & 20) is not included in this table.

TABLE 9
Identities of the new sequences with some previously described
sequences in the NCBI patent sequence database “pat”.

| | | | | Coprinopsis | Coprinopsis | Coprinopsis | Coprinopsis | |
|---|---|---|---|---|---|---|---|---|
| | Agrocybe | Agrocybe | laccaria | cinerea | cinerea | cinerea | cinerea | Coprinellus |
| | aegerita | aegerita | bicolor | okayama7#130_ | okayama7#130 | okayama7#130 | okayama7#130 | radians |
| | GM831938 | GM831940 | GM831942 | GM831944 | GM831946 | GM831948 | GM831950 | GM831952 |
| UPO 2 | 66.58 | 69.73 | 69.09 | 63.27 | 60.40 | 60.22 | 59.64 | 64.32 |
| UPO 3 | 59.79 | 61.46 | 62.63 | 58.11 | 55.91 | 56.12 | 57.14 | 62.11 |
| UPO 4 | 63.41 | 62.6 | 67.74 | 58.33 | 56.2 | 56.18 | 62.35 | 63.44 |
| UPO 5 | 63.51 | 63.69 | 94.41 | 59.68 | 56.69 | 57.95 | 60.12 | 67.84 |
| UPO 6 | 59.57 | 61.35 | 71.73 | 59.68 | 57.68 | 57.68 | 60.24 | 63.44 |
| UPO 7 | 60.27 | 60.16 | 69.33 | 57.8 | 58.6 | 54.72 | 57.1 | 63.88 |
| UPO 8 | 57.84 | 59.62 | 68.00 | 54.84 | 56.81 | 55.80 | 57.40 | 62.11 |
| UPO 9 | 26.02 | 27.31 | 28.78 | 27.68 | 27.52 | 27.04 | 25.19 | 27.67 |
| UPO 10 | 28.79 | 29.12 | 31.42 | 27.59 | 28.79 | 30.27 | 31.01 | 32.20 |
| UPO 11 | 33.33 | 32.05 | 28.96 | 29.89 | 29.25 | 30.74 | 31.52 | 30.88 |
| UPO 12 | 30.95 | 29.88 | 30.20 | 29.88 | 32.74 | 31.10 | 30.31 | 28.78 |
| UPO 13 | 27.99 | 26.67 | 28.21 | 25.38 | 29.96 | 27.90 | 29.24 | 29.47 |
| UPO 14 | 28.38 | 28.96 | 28.05 | 28.64 | 29.63 | 26.85 | 26.39 | 29.59 |
| UPO 15 | 57.72 | 57.18 | 59.25 | 56.91 | 58.02 | 56.37 | 59.39 | 64.76 |
| UPO 16 | 58.74 | 58.47 | 57.84 | 56.13 | 54.12 | 59.29 | 62.39 | 64.32 |
| UPO 17 | 28.63 | 25.68 | 26.46 | 26.06 | 24.89 | 25.68 | 27.86 | 24.64 |
| UPO 18 | 57.18 | 59.35 | 59.19 | 57.14 | 58.43 | 55.68 | 58.79 | 63.00 |

TABLE 10
Identities of the new sequences with some previously described patent
sequences

| | Coprinellus | | | | | | | |
|---|---|---|---|---|---|---|---|---|
| | radians | Sclerotinia | Aspergillus | Humicola | Myceliophthora | Pestalotiopsis | Chaetomium | Daldinia |
| | GM831954 | sclerotiorum | carbonarius | insolens | hinnulea | virgatula | yirescens | caldariorum |
| UPO 2 | 45.19 | 28.21 | 30.35 | 29.57 | 30.56 | 30.80 | 22.88 | 25.67 |
| UPO 3 | 41.35 | 28.15 | 30.59 | 29.41 | 30.35 | 28.93 | 25.38 | 27.80 |
| UPO 4 | 44.66 | 27.10 | 28.02 | 31.13 | 29.96 | 29.25 | 23.33 | 29.02 |
| UPO 5 | 45.19 | 29.92 | 30.59 | 32.12 | 30.35 | 28.23 | 26.22 | 26.25 |
| UPO 6 | 43.81 | 26.15 | 27.85 | 30.71 | 29.07 | 29.88 | 26.48 | 25.10 |
| UPO 7 | 36.63 | 29.01 | 31.37 | 32.12 | 29.30 | 30.28 | 27.56 | 29.57 |
| UPO 8 | 37.86 | 28.24 | 30.47 | 31.18 | 29.48 | 27.71 | 25.91 | 27.41 |
| UPO 9 | 26.67 | 79.93 | 42.08 | 42.19 | 40.31 | 42.86 | 28.74 | 39.84 |
| UPO 10 | 21.57 | 41.57 | 60.87 | 53.12 | 53.97 | 56.47 | 29.96 | 50.98 |
| UPO 11 | 23.08 | 41.83 | 60.47 | 53.39 | 54.76 | 57.65 | 31.47 | 52.55 |
| UPO 12 | 19.59 | 39.62 | 50.98 | 50.39 | 51.18 | 54.80 | 31.62 | 63.39 |
| UPO 13 | 27.94 | 42.58 | 49.80 | 48.87 | 61.30 | 52.78 | 31.06 | 48.85 |
| UPO 14 | 23.53 | 37.33 | 46.95 | 50.91 | 72.00 | 52.27 | 28.44 | 44.14 |
| UPO 15 | 44.12 | 23.48 | 26.56 | 28.35 | 28.29 | 27.69 | 23.40 | 24.31 |
| UPO 16 | 44.00 | 25.58 | 30.59 | 30.20 | 27.52 | 27.67 | 27.07 | 25.97 |
| UPO 17 | 21.69 | 28.14 | 35.42 | 34.25 | 30.65 | 30.04 | 71.97 | 32.03 |
| UPO 18 | 45.10 | 27.69 | 27.52 | 30.80 | 28.40 | 26.59 | 24.07 | 24.61 |

### ABTS Assays

Activity Landscapes in 100 mM Buffer

All activity landscapes formed by measured activities of individual transformants were directly done with samples (culture supernatants) from deep well plate cultivation. The slope of absorption was in all cases calculated with Microsoft Excel's “slope( )” function and is equivalent to 1.4*ΔABS*min⁻¹. The measurement was done with the plate reader.

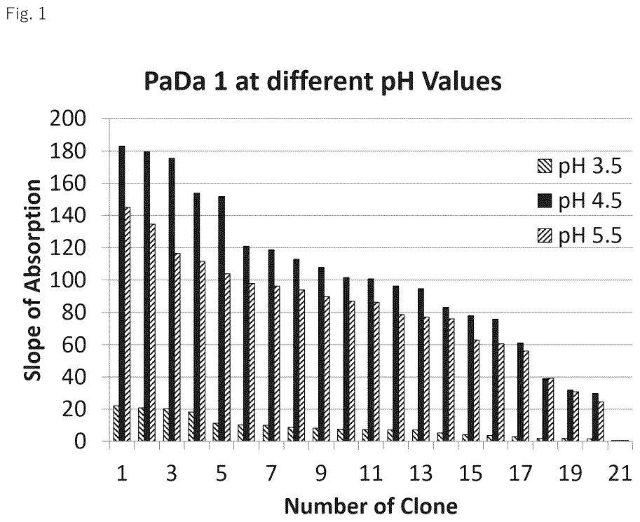

In FIG. 1 the landscapes of the PaDa1 mutant transformants (mutant of AaeUPO1) at pH 3.5, 4.5 and 5.5 were compared and showed a maximum of conversion at pH 4.5.

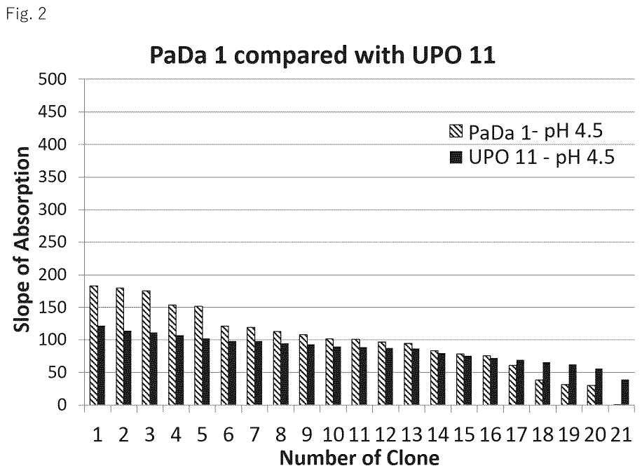

FIG. 2 shows the measurement results of AaeUPO1 mutant PaDa1 (indicated as UPO1mut) as well as UPO 11 at pH 4.5. Supernatants of UPO 1 clones converted ABTS faster than UPO 11, nonetheless also the new wt enzyme UPO 11 converted ABTS very well, indicating good expression of the recombinant protein.

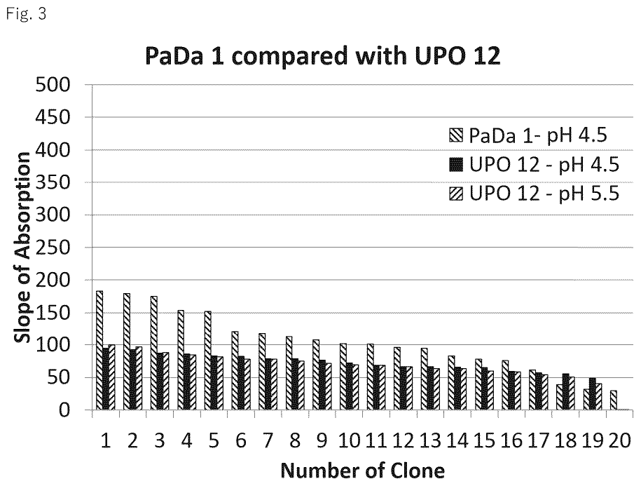

UPO 12 behaved similar to UPO 11 in ABTS peroxidase assays as shown in FIG. 3. In primary screenings UPO 11 showed a maximum conversion at pH 4.5 when tested at pH values 3.5, 4.5 and 5.5, UPO 12 in comparison showed similar behavior at pH 4.5 and 5.5, indicating a higher robustness of activity at different pH than the AaeUPO1 variant.

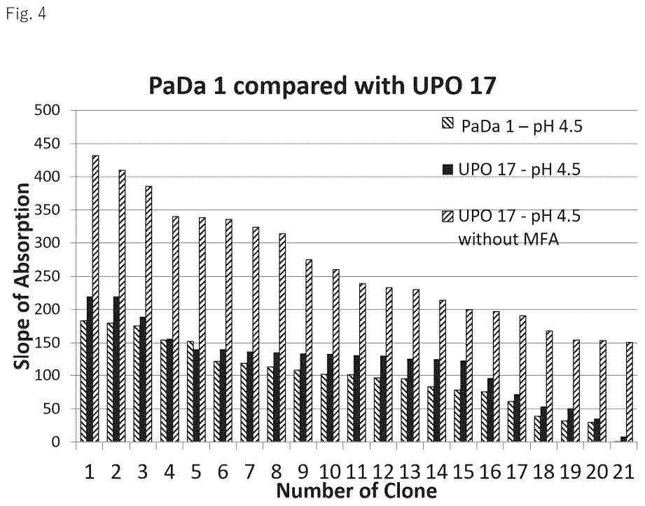

FIG. 4 shows the comparison of UPO 1, UPO 17 and UPO 17 without additional mating factor alpha-but the given native signal sequence. This given signal sequence is from Podospora anserina and increased the conversion of ABTS around 2-fold compared to the construct containing the short Saccharomyces cerevisiae's mating factor alpha signal.

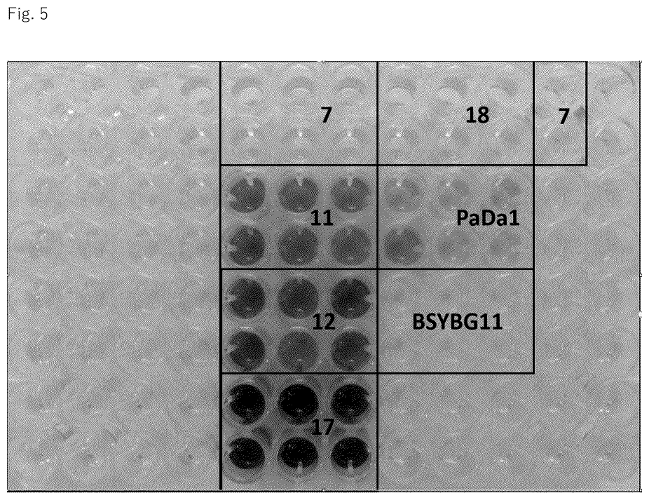

To confirm that the AaeUPO 1 with short mating factor alpha signal is converting ABTS better than with the native signal peptide was tested with given settings and the described expression system. As shown in FIG. 5 there was no mentionable activity measured for UPO 1 with the native signal peptide.

Furthermore, in this specific experiment the constructs containing PaDa 1 with mating factor alpha signal were converting ABTS poorly. Nonetheless, at least the mating factor alpha signal containing constructs were active and behaved better than the ones with native signal peptides.

Constructs with Measurable Activity in the Rescreening

TABLE 11
Results of the rescreening at pH 4.5 using the ABTS based
peroxidase assay, indicating functional expression.

| Construct | Clone | ΔABS(405 nm)/min |
|---|---|---|
| UPO 17 | 17-A0 | 349.3 |
| UPO 17 | 17-B0 | 291.7 |
| UPO 17 | 17-C0 | 285.2 |
| UPO 12 | 12-A0 | 68.8 |
| UPO 12 | 12-B0 | 68.5 |
| PaDa 1 | 1-A0 | 66.8 |
| UPO 12 | 12-C0 | 66.5 |
| UPO 11 | 11-A0 | 65.2 |
| UPO 11 | 11-B0 | 64.1 |
| UPO 11 | 11-C0 | 62.1 |
| PaDa 1 | 1-B0 | 57.9 |
| PaDa 1 | 1-C0 | 57.6 |
| CPO 19* | | 1.2 |
| CPO 19* | | 1.2 |
| CPO 19* | | 1.0 |
| UPO 3 | 3-A0 | 0.9 |
| UPO 2 | 2-A0 | 0.7 |
| UPO 15 | 15-A0 | 0.7 |
| UPO 15 | 15-B0 | 0.6 |
| UPO 5 | 5-A0 | 0.6 |
| UPO 3 | 3-B0 | 0.6 |
| UPO 15 | 15-C0 | 0.5 |
| UPO 4 | 4-A0 | 0.4 |
| UPO 2 | 2-B0 | 0.4 |
| UPO 3 | 3-C0 | 0.3 |
| UPO 4 | 4-B0 | 0.2 |
| UPO 16 | 16-A0 | 0.2 |
| Negative control/BSYBG 11 without | | 0.1 |
| integrated expression vector | | |

*Data from the primary screening

The Table shows the transformants that where most active in the rescreening with their medium change in absorption per minute, measured over 13 minutes.

As can be seen in Table 11 the rescreening of the most promising clones with the ABTS assay was successful and indicated peroxidase activity and functional expression for all tested genes. UPO17 showed higher activity than the evolved AaeUPO1 variant. Surprisingly also CPO transformants showed activity, indicating functional expression of CfuCPO by P. pastoris.

Assays after Bioreactor Cultivation

### ABTS Assay

Constructs under the PDC Promoter showed increased activity after the bioreactor cultivation compared to 96-deepwell plate cultivation, as shown in Table 12. Surprisingly the activity of UPO 17 was far lower than the activities of the UPOs 11 and 12, indicating possible enzyme instabilities caused by long term cultivation. Compared to the benchmark, AaeUPO 1 variant PaDa1, activities up to 355-fold higher were seen.

TABLE 12
The table lists the constructs cultivated in the bioreactor and the
units per milliliter unconcentrated supernatant, measured with
the ABTS assay. Furthermore, a comparison of the new UPOs
compared to the benchmark, AaeUPO1 variant PaDa1 is listed.

| | | Peroxidase activity |
|---|---|---|
| Construct | U * ml⁻¹ | compared to PaDa1 |
| PaDa l | 0.13 | 1 |
| UPO 11 | 21.55 | 166-fold |
| UPO 12 | 46.12 | 355-fold |
| UPO 17 | 6.61 | 51-fold |

Determined by the Bradford Assay the protein concentrations of the supernatant are as listed in Table 13. Protein concentrations in the supernatant from non-optimized bioreactor cultivations were equal or mostly higher than for the benchmark clone, which was also made with the new expression vectors based on the PDC promoter.

TABLE 13
Protein concentrations of the supernatant of the bioreactor
cultivations as a result of Bradford Assays. The amount of
enzyme found in the supernatant of the cultivation of the PaDa1
expression clone confirmed previous data of Molina et al (2015)

| Construct | mg * mL⁻¹ |
|---|---|
| PaDa 1 | 0.1769 |
| UPO 11 | 0.2455 |
| UPO 12 | 0.2031 |
| UPO 17 | 0.1651 |

### Naphthalene—Fast-Blue Assays

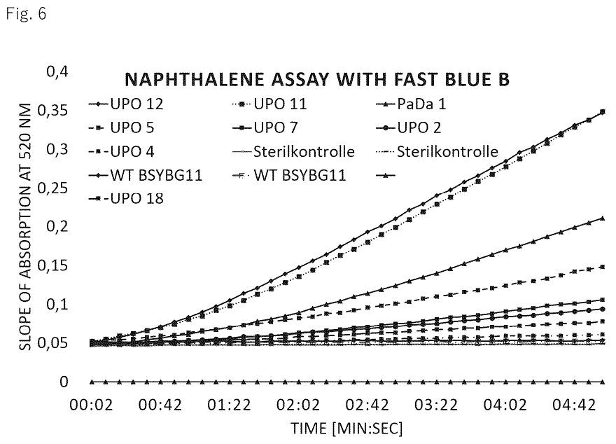

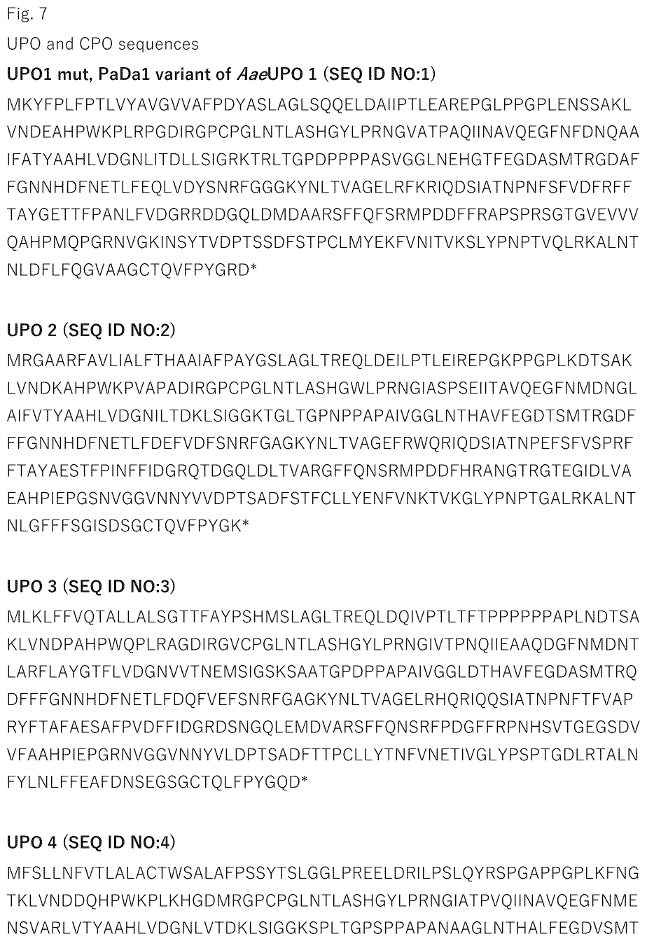

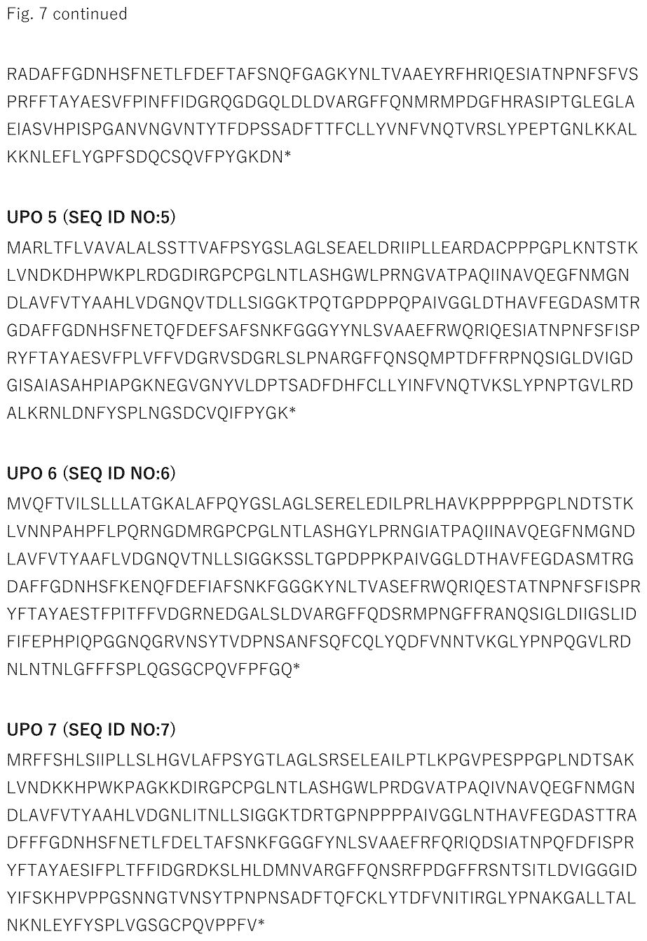

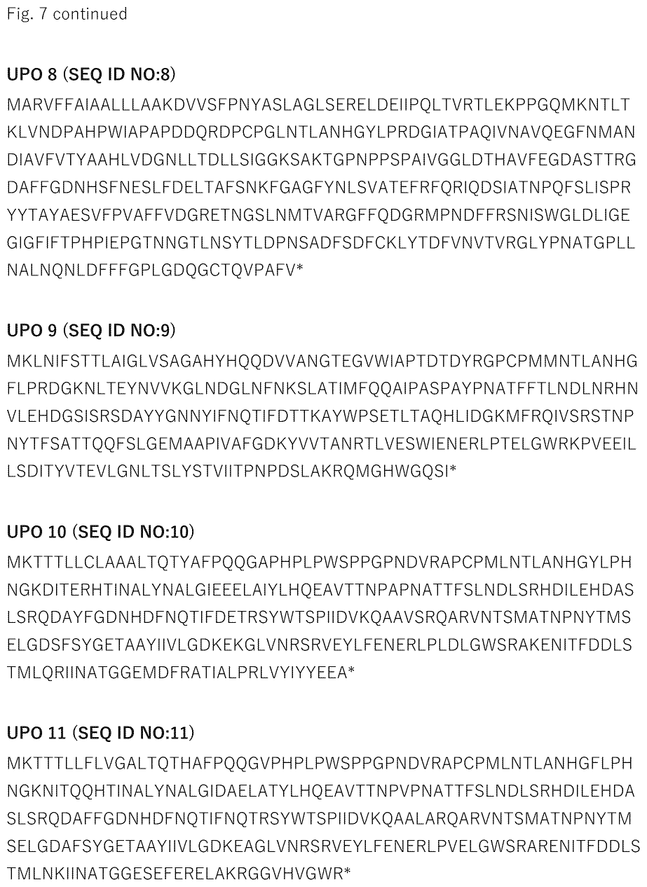

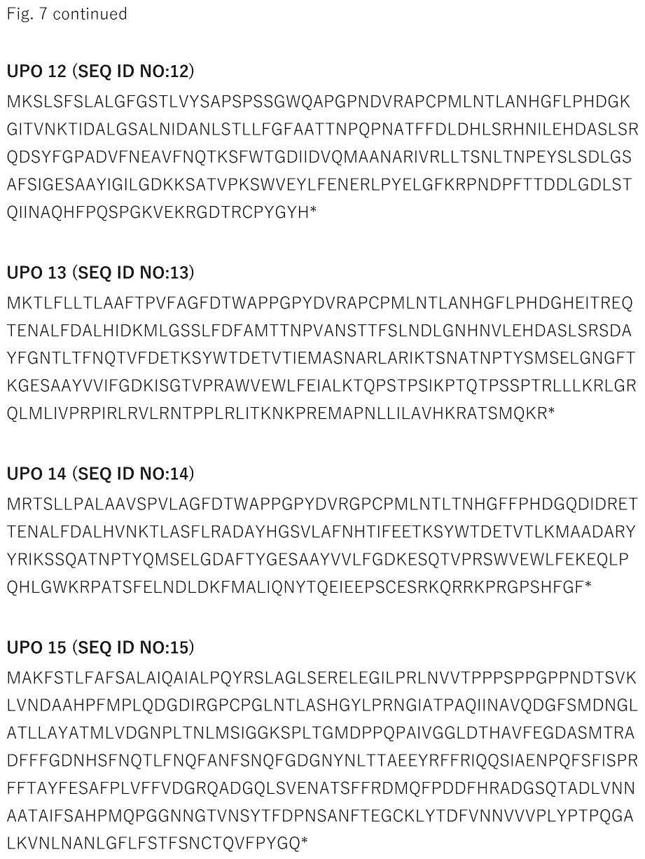

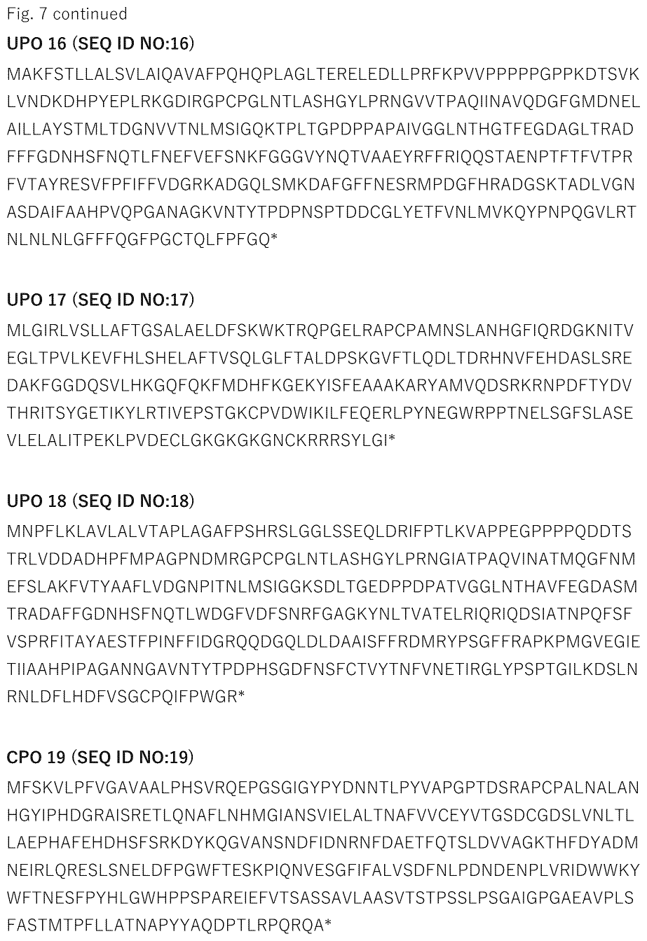

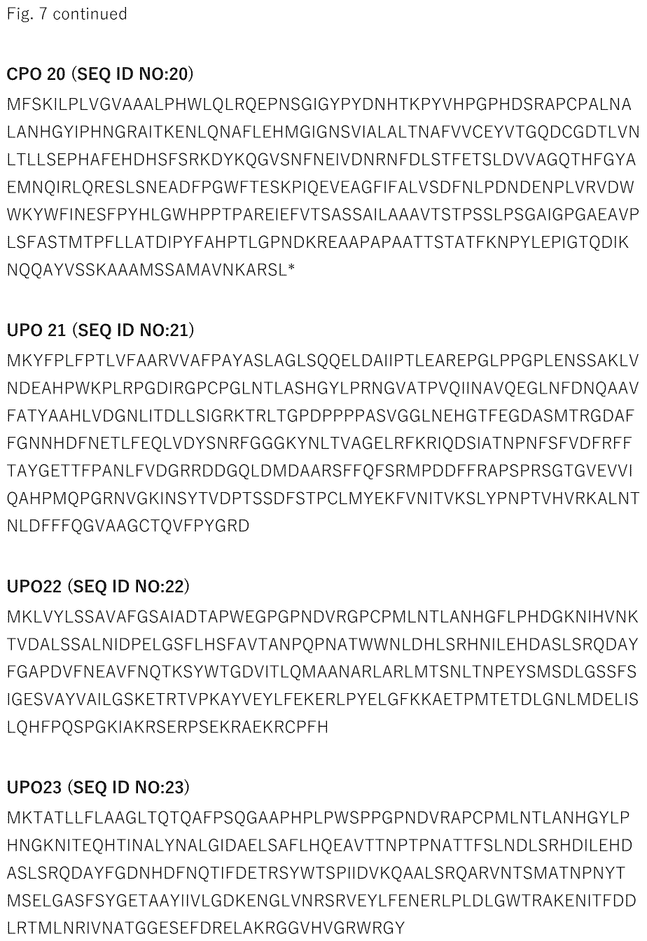

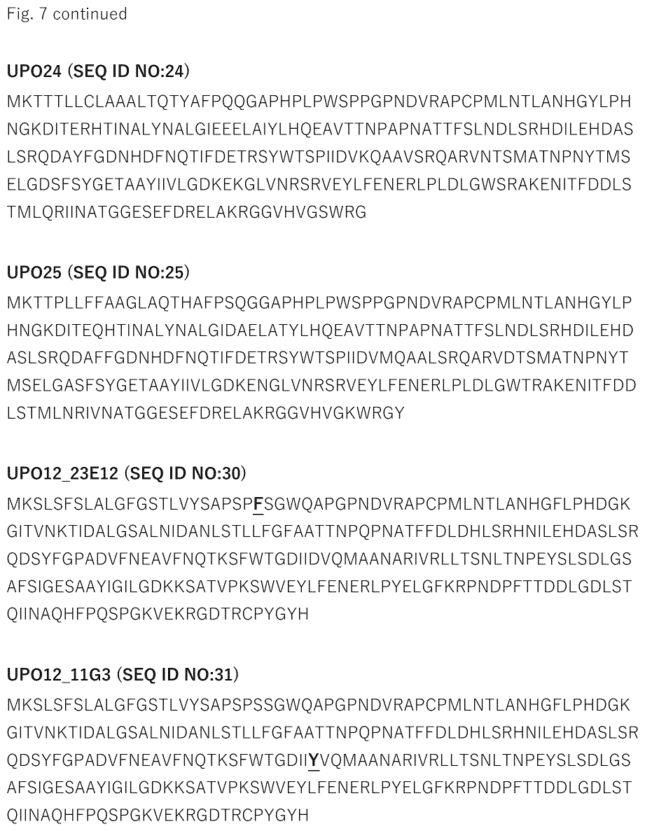

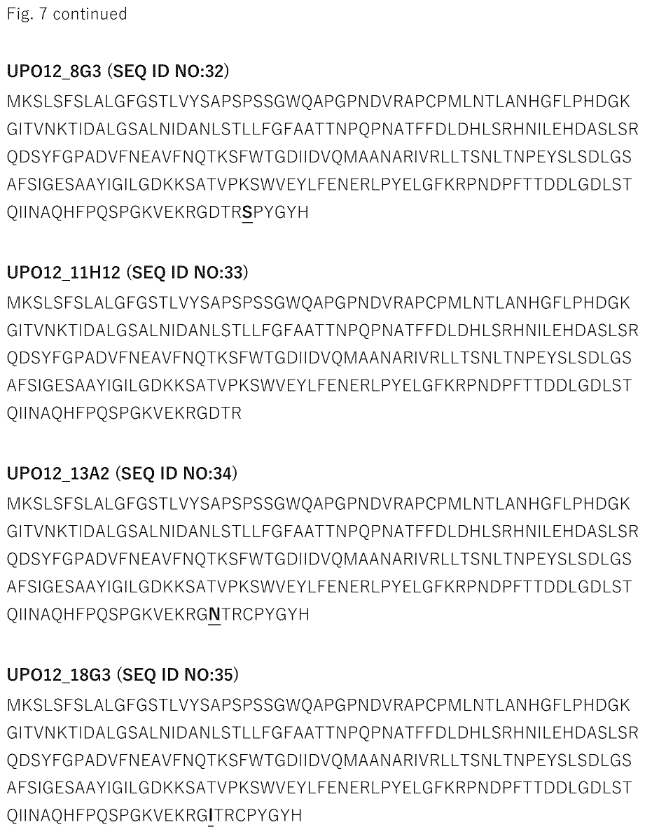

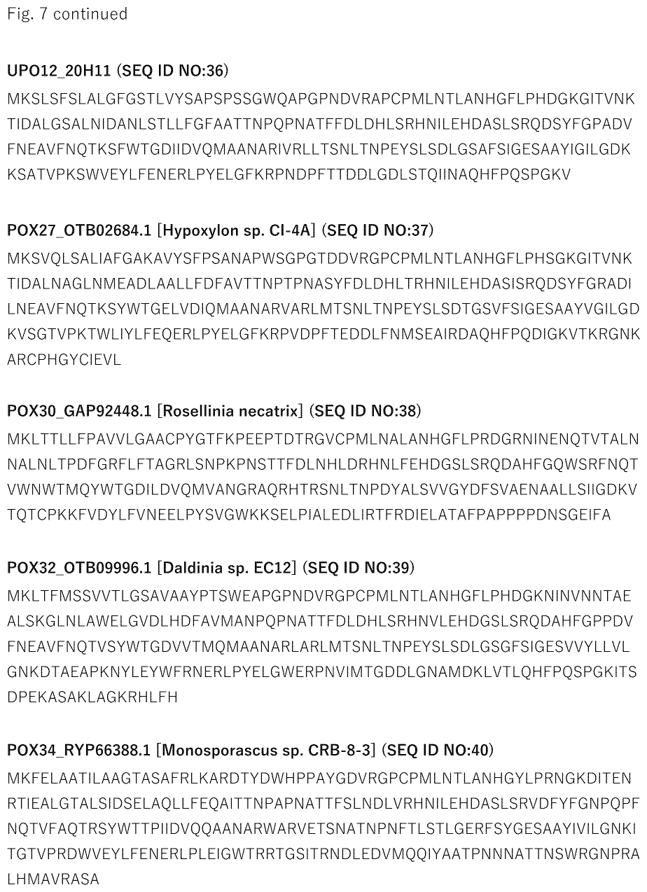

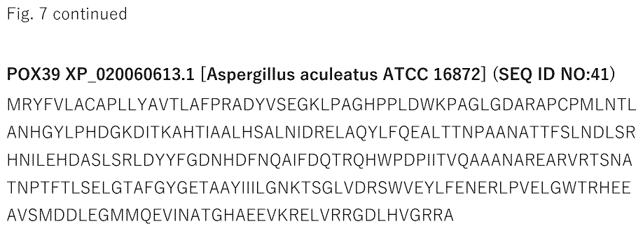

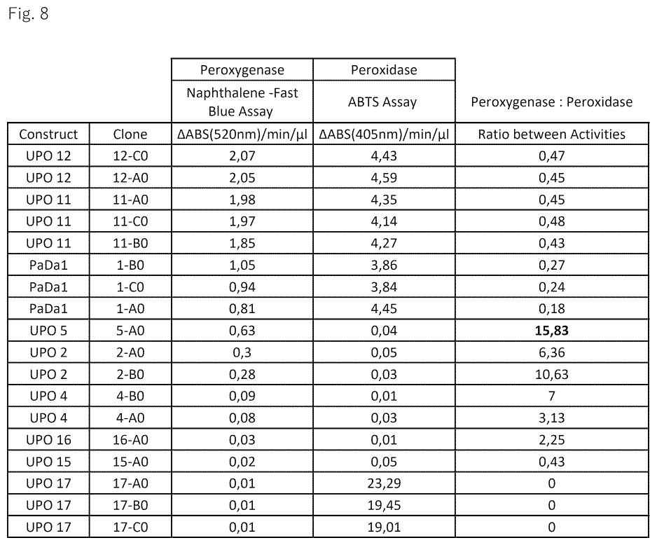

The naphthalene assay is suitable to measure peroxygenase activity. As shown in FIG. 6, there are, beneath UPO 1, six UPOs that clearly showed activity in this assay. Two of the new constructs, UPO 12 and UPO 11, converted the substrate nearly twice as fast as the known benchmark. UPO 1.

The rescreened clones that showed activity in the Naphthalene-Fast Blue-Assay are listed in Table 14, including ΔABS/min values at 520 nm.

TABLE 14
The table lists the clones that were active at the rescreening at the naphthalene-fast blue Assay and their average change
in absorption per minute at a measurement over 13 minutes.

| Construct | Clone | ΔABS(520 nm)/min |
|---|---|---|
| UPO 12 | 12-B0 | 62.7 |
| UPO 12 | 12-C0 | 62.0 |
| UPO 12 | 12-A0 | 61.6 |
| UPO 11 | 11-A0 | 59.3 |
| UPO 11 | 11-C0 | 59.2 |
| UPO 11 | 11-B0 | 55.5 |
| PaDa 1 | 1-B0 | 31.6 |
| PaDa 1 | 1-C0 | 28.2 |
| PaDa 1 | 1-A0 | 24.2 |
| UPO 5 | 5-A0 | 19.0 |
| UPO 5 | 5-C0 | 18.2 |
| UPO 5 | 5-B0 | 15.1 |
| UPO 7 | 7-A0 | 11.2 |
| UPO 2 | 2-A0 | 8.9 |
| UPO 2 | 2-B0 | 8.5 |
| UPO 7 | 7-B0 | 8.5 |
| UPO 7 | 7-C0 | 8.0 |
| UPO 2 | 2-C0 | 7.1 |
| UPO 18 | 18-A0 | 6.4 |
| UPO 18 | 18-B0 | 5.5 |
| UPO 18 | 18-C0 | 5.0 |
| UPO 4 | 4-B0 | 2.8 |
| UPO 4 | 4-C0 | 2.8 |
| UPO 4 | 4-A0 | 2.5 |
| UPO 9 | 9-1 | 1.5 |
| UPO 16 | 16-A0 | 0.9 |
| UPO 16 | 16-C0 | 0.8 |
| UPO 8 | 8-1 | 0.7 |
| UPO 9 | 9-2 | 0.7 |
| UPO 9 | 9-3 | 0.7 |
| UPO 10 | 10-1 | 0.7 |
| UPO 14 | 14-1 | 0.7 |
| UPO 15 | 15-A0 | 0.6 |
| UPO 14 | 14-2 | 0.6 |
| Negative | | 0.5 |
| control/BSYBG 11 | | |

Clear naphthalene oxidation activity was found for most tested expression clones but not all of them. For UPO14 this might be explained by the fact that the used database sequence was wrongly annotated and the used was not correct according to Kiebist et al. (2017). Surprisingly new recombinant UPOs with a higher peroxygenase/peroxidase activity ration were identified by these rescreening experiments using the ABTS and naphthalene assay, indicating the high potential of the new recombinant heme thiolate peroxygenases and the diversity of catalytic properties with diverse substrates and chemical reactions.

### Filter Assay

In the filter assay with horse radish peroxidase the promising results were obtained. A greenish zone was visible around every active colony.

### Peroxidase Plate Assay

After incubation the plates were visually inspected. The plate assay is working with HRP as positive control. All positive controls showed green zones of converted ABTS, while none of the negative controls shows any visible conversion.

For the tested UPOs the plate assays did not show changes after one day. Therefore, the plates were stored for more than a week in the fridge. Surprisingly the color of the plate with the pH 4.5 buffer turned green at those plates, while the plate with the pH 6.0 buffer showed no changes. Color changes were expected for pH 4.5, because most UPOs are active at this pH value.

UPO12 Variants with Increased Peroxygenase Activity

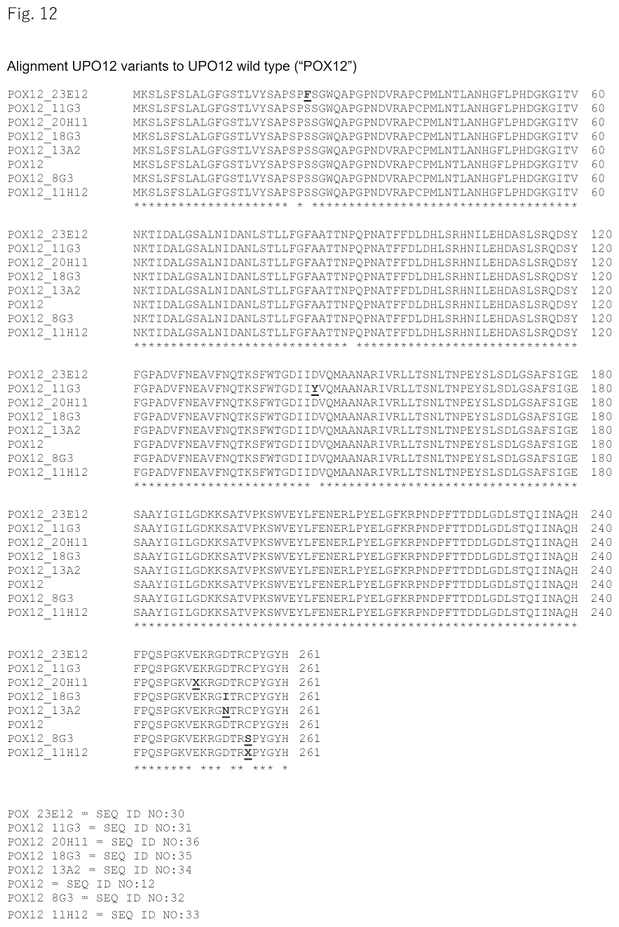

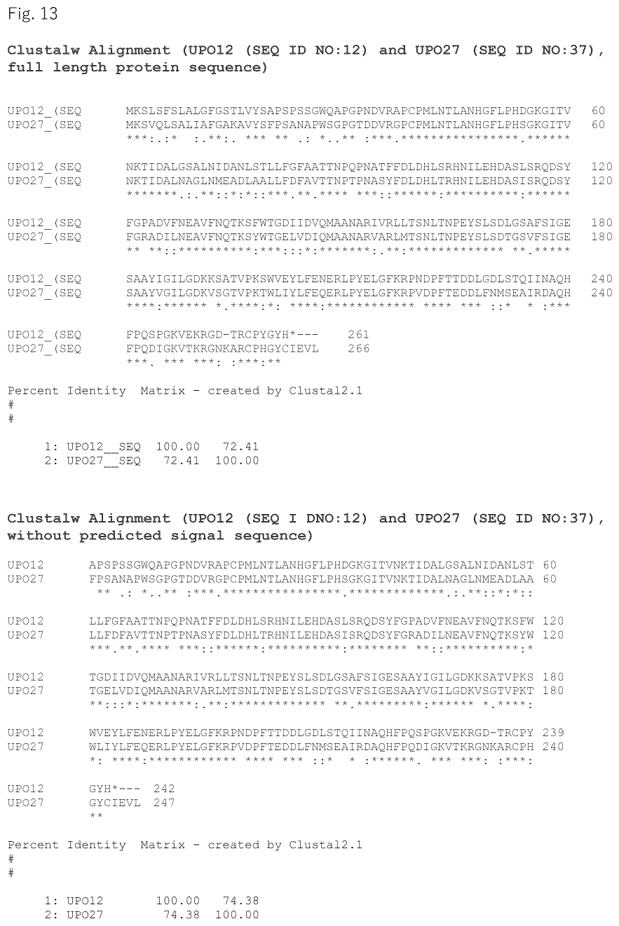

In this Example, a UPO12 mutant library was screened for superior variants of UPO12 using ABTS, naphthalene and 2,6-DMP as substrates. Surprisingly, the largest group of improved variants were found to have a mutation at the C-terminus of the POX12 (UPO12) protein sequence (see FIG. 12).

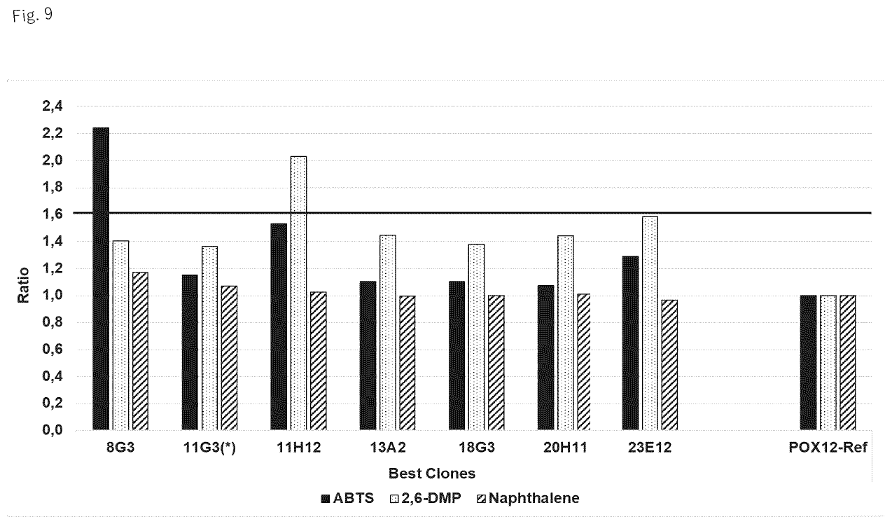

Multiple variants of UPO12 (SEQ ID NO:12) were identified, also referred to herein as POX12, that showed improved activity on one or more of the tested substates (ABTS, 2,6-DMP, naphthalene) or altered substrate profiles compared to UPO12 wild type (i.e. variants 23E12 (SEQ ID NO:30), 11G3 (SEQ ID NO:31), 8G3 (SEQ ID NO:32), 11H12 (SEQ ID NO:33), 13A2 (SEQ ID NO:34), 18G3 (SEQ ID NO: 35) and 20H11 (SEQ ID NO:36)) (see FIG. 9).

As for UPO12, the corresponding genes of the 11 variants were cloned into the pBSY5S1Z integrative expression vector (containing a FMD promoter fragment of Hansenula polymorpha) via BioXP™ after codon optimization and replacement of their native secretion signals by the alpha factor secretion signal variant (MataD, a deletion variant of the S. cerevisiae mating factor aslpha signal sequence). The expression vector was introduced in P. pastoris for secretion of the variants.

Best results were obtained for variants 8G3 and 11H12. Variant 8G3 (C256S) had an amino acid exchange from cysteine (C) to serine(S) at position 256 which is just 5 amino acids prior the end of the protein. This exchange resulted in a doubling of peroxidase activity, i.e., twice as high activity on ABTS. Variant 8G3 also showed a 1.4-fold improvement on 2,6-DMP and 1.2-fold improvement on naphthalene. Also clone 11H12 showed twice as high activity on 2,6-DMP and a 1.5-fold higher activity on ABTS compared to the UPO12 reference clone. In agreement with the results from clone 8G3 and very surprising, also clone 11H12, showed a mutation at the very same position (C256X); however, a stop codon instead of cysteine.

Similarly, variants 20H11 (E249X), 13A2 (D253N), 18G3 (D253I), showed a C-terminal modification associated with higher activity, and showed an increase of at least 1.4-fold on 2,6-DMP.

Interestingly, variant 23E12 (S24F) also had an amino acid exchange from polar serine to large hydrophobic phenylalanine just two amino acids further resulting in 1.3-fold and 1.6-fold higher activity on ABTS and 2,6-DMP, respectively.

Activities of UPO12 variants and their corresponding amino acid mutations compared to wild type UPO12 are summarized in FIG. 9 and Table 15.

TABLE 15
Mutation grouping

| N-terminal | Middle/C-terminal | C-terminal |
|---|---|---|
| 23E12 (S24F) | 11G3(D145YY)⁽*⁾ | 8G3 (C256S) |
| | | 11H12 (C256X) |
| | | 13A2 (D253N) |
| | | 18G3 (D253I) |
| | | 20H11 (E249X) |

UPO12 variants amino acid sequence mutations. The variants are listed in groups related to the position of the mutation (N-terminal, middle, C-terminal or signal sequence), some clones were identified as WT for others sequencing was unambiguous (“n.s.r.” = no sequencing results).
^{(*)}This clone did not show activity on any of the three substrates (ABTS, 2,6-DMP, naphthalene) in shake flask.

### Identification of New Highly Active Peroxygenase Biocatalysts

In this Example, novel peroxygenases were identified by BLAST search using the UPO12 protein sequence (SEQ ID NO:12) as reference. Identified candidates were expressed in P. pastoris and screened for activity on ABTS, naphthalene and 2,6-DMP.

Using the UPO12 wild type amino acid sequence (SEQ ID NO: 12) and the online BLAST tool from NCBI, 17 homologous enzymes containing the PCP-motif were identified. As for UPO12 the corresponding genes were cloned into the pBSY5S1Z integrative expression vector (containing an FMD promoter fragment of Hansenula polymorpha) via BioXP™ after codon optimization and replacement of their native secretion signals by the alpha factor secretion signal variant (MataD, a deletion variant of the S. cerevisiae mating factor alpha signal sequence).

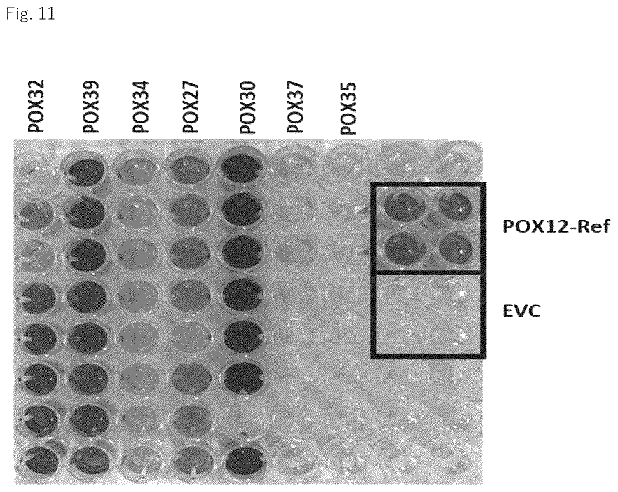

After P. pastoris transformation, screening of transformants identified four new UPOs with high activity on ABTS (POX27 (SEQ ID NO:37), POX32 (SEQ ID NO: 39), POX34 (SEQ ID NO:40), POX39 (SEQ ID NO:41), see FIG. 11), three of them were also found active on 2,6-DMP and naphthalene (POX27, POX32, POX39). These UPOs were also studied in a reaction with ABTS using 2 mM H₂O₂ (8-fold concentration).

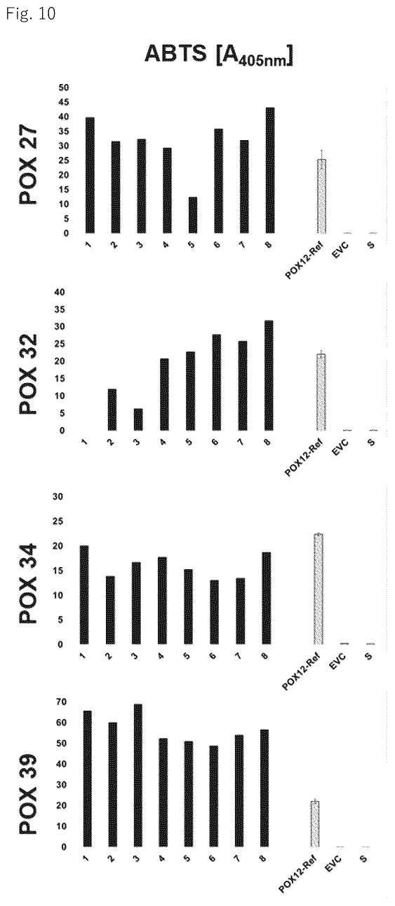

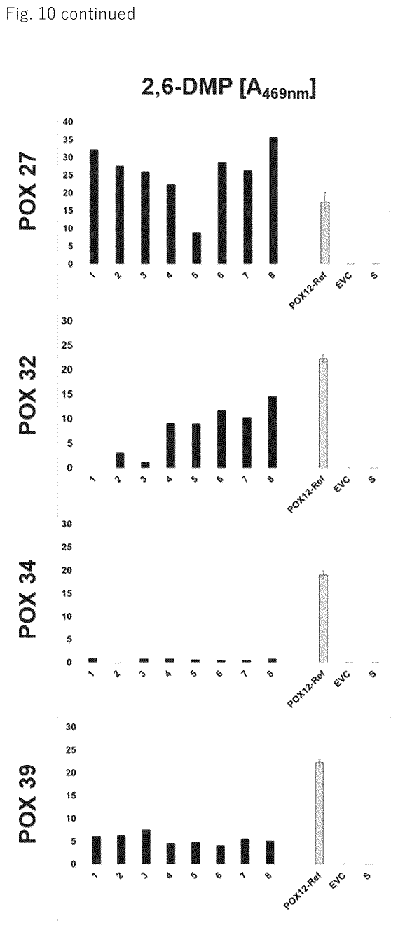

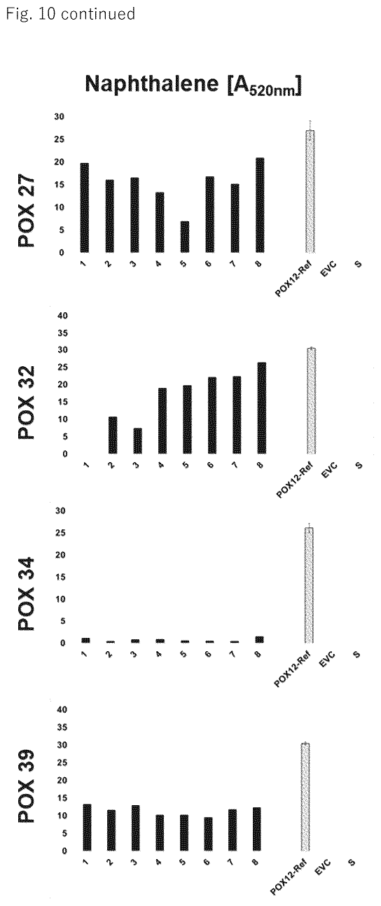

Surprisingly, another highly active UPO showing significantly higher activity on ABTS than the reference was identified (POX30 (SEQ ID NO:38), see FIG. 11). Novel peroxygenases showing significant activity on one or more of the tested substrates are summarized in FIG. 10 and FIG. 11.

### REFERENCES

Babot, Esteban D. et al. 2013. “Oxyfunctionalization of Aliphatic Compounds by a Recombinant Peroxygenase from Coprinopsis Cinerea.” Biotechnology and Bioengineering 110 (9): 2323-32
Bormann, S. et al. “Specific oxyfunctionalisations catalysed by peroxygenases: opportunities, challenges and solutions.” Catal. Sci. Technol. volume 5, issue 4, P2038-2052 2015
Faiza et al., BMC “New insights on unspecific peroxygenases: superfamily reclassification and evolution” Evol Biol. 2019 Mar. 13; 19 (1): 76.
Grôbe, Glenn et al. 2011. “High-Yield Production of Aromatic Peroxygenase by the Agaric Fungus Marasmius Rotula.” AMB Express 1 (1): 1-11.
Kiebist et al. “A Peroxygenase from Chaetomium globosum Catalyzes the Selective Oxygenation of Testosterone.” Chembiochem. 2017 Mar. 16; 18 (6): 563-569
Molina-Espeja et al., “Directed evolution of unspecific peroxygenase from Agrocybe aegerita” Appl Environ Microbiol. 2014 June; 80 (11): 127-143.
Molina-Espeja, Patricia et al. 2015. “Tandem-Yeast Expression System for Engineering and Producing Unspecific Peroxygenase.” Enzyme Microb Technol. 2015 June; 73-74:29-33.
Morawski, Birgit et al. 2000. “Functional Expression of Horseradish Peroxidase in Saccharomyces Cerevisiae and Pichia Pastoris.” Protein Engineering, Design and Selection 13 (5): 377-84. https://academic.oup.com/peds/article-lookup/doi/10.1093/protein/13.5.377.
Pecyna, Marek J. et al. 2009. “Molecular Characterization of Aromatic Peroxygenase from Agrocybe Aegerita.” Applied Microbiology and Biotechnology 84 (5): 885-97.
Pütter, J., & Becker, R. (1983). Peroxidase. In H. U. Bergmeyer, J. Bergmeyer, & M. Grassl (Ed.), Methods of enzymatic analysis (pp. 286-293). Weinheim: Verlag Chemie. Yuan, Z. Y., & Jiang, T. J. (2002). Horseradish peroxidase. In J. R. Whitaker, A. G. J. Voragen, & D. W. S. Wong (Ed.), Handbook of food enzymology (pp. 403-411). New York: CRC Press.
Zámocký, Marcel et al., “Independent Evolution of Four Heme Peroxidase Superfamilies.” Arch Biochem Biophys. 2015 May 15; 574:108-19.

1. A method for producing a polypeptide having peroxygenase activity, comprising:
a. cultivating a yeast cell in a medium for the production of said polypeptide, wherein the yeast cell comprises a polynucleotide comprising a nucleic acid sequence encoding said polypeptide operably linked to a derepressed and methanol-independent promoter sequence which is functional in methylotrophic yeasts, and
b. isolating said polypeptide from the cultivation medium.

2. The method of claim 1, wherein said promoter is an engineered or synthetic promoter variant.

3. The method of claim 1, wherein said promoter is an engineered or synthetic promoter.

4. The method of any one of claim 1, wherein expression of the polypeptide is increased by co-expression of a helper protein.

5. The method of claim 4, wherein the helper protein is protein disulfide isomerase.

6. The method of any one of claim 1, wherein said yeast cell is a Pichia pastoris (Komagataella phaffii) cell.

7. The method of one of claim 1, wherein said polypeptide is obtained in a yield of about 50 mg/L, or of about 100 mg/L, or of about 250 mg/L.

8. The method of one of claim 1, wherein said polypeptide is obtained in supernatant of the cultivation medium in a titer of about 300 mg/L, or of about 0.5 g/L, or of about 1 g/L.

9. A polypeptide having peroxygenase activity produced according to the method of claim 1.
